(19) United States
(12) Patent Application Publication
(10) Pub. No.: US 2022/0112557 A1
(43) Pub. Date: Apr. 14, 2022

(54) SYSTEM AND METHODS FOR MONITORING ADOPTIVE CELL THERAPY CLONALITY AND PERSISTENCE

(71) Applicant: Iovance Biotherapeutics, Inc., San Carlos, CA (US)

(72) Inventors: Cecile Chartier-Courtaud, Palo Alto, CA (US); Viktoria Gontcharova, Redwood City, CA (US)

(21) Appl. No.: 17/420,787

(22) PCT Filed: Jan. 10, 2020
(86) PCT No.: PCT/US2020/013095

§ 371 Date: Jul. 6, 2021

Related U.S. Application Data
(60) Provisional application No. 62/826,209, filed on Mar. 29, 2019.
(60) Provisional application No. 62/790,898, filed on Jan. 10, 2019.
(60) Provisional application No. 62/896,354, filed on Sep. 5, 2019.

(51) Int. Cl. C12Q 1/6881; C12Q 1/6886; A61K 35/17

(52) U.S. Cl. CPC C12Q 1/6881; C12Q 2600/106; A61K 35/17; C12Q 1/6886

(57) ABSTRACT

Methods and systems for identifying clinically effective population of tumor infiltrating lymphocytes are disclosed. Also disclosed are methods for identifying persistent unique clones derived from adoptive cell therapy products.

### CROSS-REFERENCE TO RELATED APPLICATIONS

This application claims priority to U.S. Provisional Patent Application No. 62/790,898, filed on Jan. 10, 2019, U.S. Provisional Patent Application No. 62/826,209, filed on Apr. 8, 2019, and U.S. Provisional Patent Application No. 62/896,354, filed on Sep. 5, 2019, each of which is hereby incorporated by reference in its entirety.

### FIELD OF THE INVENTION

The invention described herein relates generally to identifying a clinically effective population of tumor infiltrating lymphocytes, and more particularly, but not exclusively, to identifying a clinically effective population of T-cells.

### BACKGROUND OF THE INVENTION

While general methods for profiling T-cell receptors in cancer patients are known, e.g., Kirsch et al., Molecular Oncology, 9:2063-2070 (2015), these methods have proven to be of little utility in assessing potential clinical response to cancer therapies. In particular, such profiling studies produce myriad data with no clear conclusions about the impact of a particular therapy on T-cell diversity and the relationship of the measured diversity on clinical response and/or clinical outcome, e.g., Snyder et al., PLOS Medicine, 14:e1002309 (2017). This challenge is particularly acute for adoptive cell therapies where the therapy itself changes the patient's immune repertoire, e.g. Milone and Bhoj, Molecular Therapy: Methods & Clinical Development 8:210-221 (2018).

In particular, in adoptive cell therapy treatments, the identification of the clinically relevant sub-population of cells within a heterogeneous polyclonal therapeutic population of cells is a critical problem. Presented herein are methods and systems for identifying clinically effective populations of tumor infiltrating lymphocytes (TILs), in particular by identifying the T cell receptor (TCR) complementarity determining region 3 (CDR3)-encoding nucleic acid sequence clones constituting the TCR CDR3 clonal diversity in order to facilitate identification of clinically effective population of tumor infiltrating lymphocytes (TILs).

### SUMMARY OF THE INVENTION

The present invention is directed to methods and systems for using various nucleic acid sequence-based profiles of lymphocyte clonal diversity to identify clinically effective populations of tumor infiltrating lymphocytes. The invention is exemplified in a number of implementations and applications, some of which are summarized below and throughout the specification.

In one aspect, the invention is directed to a method for identifying a clinically effective population of tumor infiltrating lymphocytes (TILs), the method comprising: identifying a clinically effective population of tumor infiltrating lymphocytes (TILs) in a therapeutic population of TILs administered to a subject, the method comprising:

(i) identifying the T cell receptor (TCR) complementarity determining region 3 (CDR3)-encoding nucleic acid sequence clones constituting the TCR CDR3 clonal diversity of a therapeutic population of TILs;

(ii) identifying the TCR CDR3-encoding nucleic acid sequence clones constituting the TCR CD3 clonal diversity of a first population of peripheral blood mononuclear cells (PBMCs), wherein the first population of PBMCs is isolated from a subject at least 14-days after the therapeutic population of step (i) is administered to said subject;

(iii) for each unique TCR CDR3-encoding nucleic acid sequence clone identified in step (ii), determining the frequency of such unique TCR CDR3 clone in each of the therapeutic population of TILs and the first population of PBMCs;

(iv) sorting the unique TCR CDR3-encoding nucleic acid sequence clones identified in step (ii) from highest frequency to lowest frequency for each of the therapeutic population of TILs and the first population of PBMCs; and,

(v) selecting the ten highest frequency unique TCR CDR3-encoding nucleic acid sequence clones from the first population of PBMCs sorted in step (iv), wherein the TILs expressing such clones in the therapeutic population of TILs constitute a clinically effective population of TILs, thereby identifying the clinically effective population of TILs.

In another aspect, the invention is directed to a method for determining the persistence and activity of T cell receptor (TCR) complementarity determining region 3 (CDR3)-encoding nucleic acid sequence clones in tumor infiltrating lymphocytes (TILs) in a therapeutic population of TILs administered to a subject, the method comprising:

(a) identifying the TCR CDR3-encoding nucleic acid clones constituting the TCR CDR3 clonal diversity of a therapeutic population of TILs;

(b) identifying the TCR CDR3-encoding nucleic acid sequence clones constituting the TCR CDR3 clonal diversity of a first population of peripheral blood mononuclear cells (PBMCs), wherein the first population of PBMCs is isolated from a subject at least 14-days after the therapeutic population of step (a) is administered to said subject;

(c) for each unique TCR CDR3-encoding nucleic acid sequence clone identified in step (b), determining the frequency of such unique TCR CDR3-encoding nucleic acid sequence clone in each of the therapeutic population of TILs and the first population of PBMCs; and

(d) for each unique TCR CDR3-encoding nucleic acid sequence clone identified in step (b), comparing the frequency of the TCR CDR3-encoding nucleic acid sequence clone in the first population of PBMCs to the frequency of the TCR CDR3-encoding nucleic acid sequence clone in the therapeutic population of TILs to determine the persistence and activity of TCR CDR3-encoding nucleic acid sequence clones in the therapeutic TIL population administered to the subject.

In another aspect, the invention is directed to a system for identifying a clinically effective population of tumor infiltrating lymphocytes (TILs), the system comprising: memory; one or more processors; and one or more modules stored in memory and configured for execution by the one or more processors, the modules comprising instructions for:

(a) identifying the T cell receptor (TCR) complementarity determining region 3 (CDR3)-encoding nucleic acid sequence clones constituting the clonal diversity of a therapeutic population of TILs;

(b) identifying the T cell receptor (TCR) complementarity determining region 3 (CDR3)-encoding nucleic acid sequence clones constituting the clonal diversity of a first population of peripheral blood mononuclear cells (PBMCs), wherein the first population of PBMCs is isolated from a subject at least 14-days after the therapeutic population of step (a) is administered to said subject;

(c) for each unique TCR CDR3-encoding nucleic acid sequence clone identified in step (b), determining the frequency of such unique TCR CDR3 clone in each of the therapeutic population of TILs and the first population of PBMCs;

(d) sorting the unique TCR CDR3-encoding nucleic acid sequence clones identified in step (b) from highest frequency to lowest frequency for each of the therapeutic population of TILs and the first population of PBMCs; and,

(e) selecting the ten highest frequency unique TCR CDR3-encoding nucleic acid sequence clones from the first population of PBMCs sorted in step (d), wherein the TILs expressing such clones in the therapeutic population of TILs constitute a clinically effective sub-population of TILs, thereby identifying the clinically effective population of TILs.

In further embodiments of the above methods and systems, DNA, RNA, or both DNA and RNA is used to determine the CDR3 clonal diversity of a therapeutic population of TILs or the CDR3 clonal diversity of PBMCs.

In another aspect, the invention is directed to a method of using TCR repertoire analysis to determine TIL production process comparability, the method comprising:

determining a first set of unique CDR3 sequences expressed by the TILs in the first sample;

determining a second set of unique CDR3 sequences expressed by the TILs in the second sample;

determining (i) a ratio of the number of unique CDR3 sequences occurring in both the first set and the second set to a number of the unique CDR3 sequences in the first set, and/or (ii) a ratio of the number of unique CDR3 sequences occurring in both the first set and the second set to a number of the unique CDR3 sequences in the second set; and

based on said ratio, determining the comparability of the TILs in the first sample and the TILs in the second sample.

In further embodiments of the above methods, the TILs of the first sample are produced at a first site, and wherein the TILs of the second sample are produced at a second site.

In further embodiments of the above methods, the number of unique CDR3 sequences in the first set correlates to the therapeutic efficacy of the TILs in the first sample.

In further embodiments of the above methods, the number of unique CDR3 sequences in the second set correlates to the therapeutic efficacy of the TILs in the second sample.

In further embodiments of the above methods, the first sample and the second sample are derived from the same sample.

In further embodiments of the above methods, at least one of the first sample and the second sample are obtained from a subject having a solid tumor cancer.

In further embodiments of the above methods, the solid tumor cancer is selected from the group consisting of melanoma (including uveal melanoma), ovarian cancer, cervical cancer, non-small-cell lung cancer (NSCLC), lung cancer, bladder cancer, breast cancer, pancreatic cancer, colorectal cancer, stomach cancer, squamous cell carcinoma, basal cell carcinoma, cancer caused by human papilloma virus, head and neck cancer (including head and neck squamous cell carcinoma (HNSCC)), brain cancer glioblastoma (including GBM), gastrointestinal cancer, renal cancer, and renal cell carcinoma. In further embodiments of the above methods, the solid tumor cancer is melanoma. In further embodiments of the above methods, the solid tumor cancer is cervical cancer.

In further embodiments of the above methods, the TILS in the first sample and the TILs in the second sample are post-rapid expansion process (REP) TILs.

In further embodiments of the above methods, the ratio having a value of about 0.4 or greater, about 0.42 or greater, about 0.44 or greater, about 0.46 or greater, about 0.48 or greater, about 0.50 or greater, about 0.52 or greater, about 0.54 or greater, about 0.56 or greater, about 0.58 or greater, or about 0.60 or greater indicates comparability between the first sample and the second sample.

In further embodiments of the above methods, the CDR3 clonal diversity of TILs in the first sample and/or the second sample is determined by DNA sequencing. In further embodiments of the above methods, the CDR3 clonal diversity of TILs in the first sample and/or the second sample is determined by RNA sequencing.

In further embodiments of the above methods, the first set of unique CDR3 sequences consists of a given number of CDR3 sequences expressed at the highest frequency by the TILs in the first sample. In further embodiments of the above methods, the second set of unique CDR3 sequences consists of a given number of CDR3 sequences expressed at the highest frequency by the TILs in the second sample.

In further embodiments of the above methods, the given number is selected from the group consisting of about 5, about 10, about 15, about 20, about 25, about 50, about 75, about 100, about 150, about 200, about 250, about 300, about 350, about 400, about 450, about 500, and greater than about 500.

In further embodiments of the above methods, the given number correlates to greater than about 10%, greater than about 20%, greater than about 30%, greater than about 40%, greater than about 50%, greater than about 60%, greater than about 70%, greater than about 80%, greater than about 90%, or about 100% of a total number of sequences in one or both of the first set of unique CDR3 sequences and the second set of unique CDR3 sequences.

In further embodiments of the above methods, the given number is between about 10 and about 20. In further embodiments of the above methods, the given number correlates to about 40% of a total number of sequences in one or both of the first set of unique CDR3 sequences and the second set of unique CDR3 sequences. In further embodiments of the above methods, the given number is between about 300 and about 400. In further embodiments of the above methods, the given number correlates to about 80% of a total number of sequences in one or both of the first set of unique CDR3 sequences and the second set of unique CDR3 sequences.

These above-characterized aspects, as well as other aspects, of the present invention are exemplified in a number of illustrated implementations and applications, some of which are shown in the figures and examples, and characterized in the claim section that follows. However, the above summary is not intended to describe each illustrated embodiment or every implementation of the invention.

### BRIEF DESCRIPTION OF THE DRAWINGS

The foregoing summary, as well as the following detailed description of the invention, will be better understood when read in conjunction with the appended drawings.

FIG. 1 illustrates an overview of Example 1.

FIG. 2 illustrates the Shannon Entropy of the unique CDR3 (uCDR3) clonotypes for patients responding to TIL therapy and patients not responding to TIL therapy.

FIG. 3 illustrates a data table showing clonal persistence, as determined by methods of the invention, 42 days after administration of a TIL population to clinical trial patients.

FIG. 4 illustrates clonal diversity of responding and non-responding patient groups and the persistence of uCDR3 clones from the administered TIL population in patient PBMCs 42 days after infusion.

FIG. 5 illustrates that the shared uCDR3 clones are derived from the administered TIL product.

FIG. 6 illustrates a plot of the by-subject persistent clones derived from the administered TIL product.

FIG. 7 illustrates the uCDR3 clonal diversity present in seven responding patients.

FIG. 8 illustrates a summary of the results from Example 1.

FIG. 9 illustrates a logic flow chart of a uCDR3 clonotype analysis according to methods of the invention.

FIG. 10 illustrates a 22-day process for harvesting, expanding, and preparing for infusion TIL products of non-selected polyclonal autologous T-cells.

FIG. 11 illustrates TIL unique CDR3 sequences by response in Cohort 2. FIG. 11B illustrates TIL Shannon Entropy (index) by response in Cohort 2.

FIG. 12A illustrates the number of TIL clones in TIL product as compared with a matched sample of PBMC at Day 42. FIG. 12B illustrates percentage of TIL clones at TIL collection (100%), Day 42 (55.33%), and circulating pre-infusion (15.13%).

FIG. 13 illustrates a plot of the by-subject persistent clones derived from the administered TIL product. Each dot represents the rank as a percentage within the TIL product for each of the top ranking clones. The most frequent clones in the TIL product correspond to the lowest values; similarly, the lowest frequency clones in the TIL product correspond to values closer to 100. Persisting clones were identified at both high and low levels in the TIL product. uCDR3 clones represented at either high or low frequencies in the TIL product could persist for at least 6 weeks post-infusion.

FIG. 14 illustrates a shared uDCR3 in TIL (%) by response in Cohort 2. A slight correlation is shown between matched TIL product and D42 PMBC samples. The number of clones detected in both the TIL product and the D42 PMBC samples were divided by the number of unique CDR3 clones in the TIL products to determine a percentage of persisting clones and provide a measure of overlap between the composition of the infusion product and T-cells circulating in vivo. Results shown as box plots.

FIG. 15A illustrates CDR3s across all patients tested. Each of the white lines represent each individual clone stacked in columns above their respective subject and ordered based on descending number of subjects containing that clone. FIG. 15B is a table showing CDR3 sequences found in greater than 4 patients. The number of subjects with common clones, the number of clones in each of those groups, and the number of non-tumor related clones, is shown.

FIG. 16 illustrates a diagram of manufacturing site and test site comparability.

FIGS. 17A-C illustrate a summary of the Stage 3 comparability of clinical sample iREP data and site-specific sample iREP data obtained using commercially available iRepertoire technology (Huntsville, Ala.), including a percentage of unique CDR3 sequences shared between samples being compared.

FIG. 18 illustrates a summary of the comparability of peripheral blood lymphocyte iREP data obtained using commercially available iRepertoire technology (Huntsville, Ala.), including a percentage of unique CDR3 sequences shared between samples being compared.

FIG. 19 illustrates a summary of the Stage 1 comparability of site-specific sample iREP data obtained using commercially available iRepertoire technology (Huntsville, Ala.), including a percentage of unique CDR3 sequences shared between samples being compared.

FIG. 20 illustrates a summary of the comparability of unrelated tumor infiltrating lymphocyte sample iREP data obtained using commercially available iRepertoire technology (Huntsville, Ala.), including a percentage of unique CDR3 sequences shared between samples being compared.

FIG. 21 illustrates a summary of site-specific sample iREP data of samples obtained from patients having chronic lymphocytic leukemia, the data obtained using commercially available iRepertoire technology (Huntsville, Ala.).

FIG. 22 illustrates a summary of the comparability of unrelated peripheral blood lymphocyte sample iREP data obtained using commercially available iRepertoire technology (Huntsville, Ala.), including a percentage of unique CDR3 sequences shared between samples being compared.

FIG. 23 provides a table of CDR3 sequences, sorted by the percentage at which each clone appears.

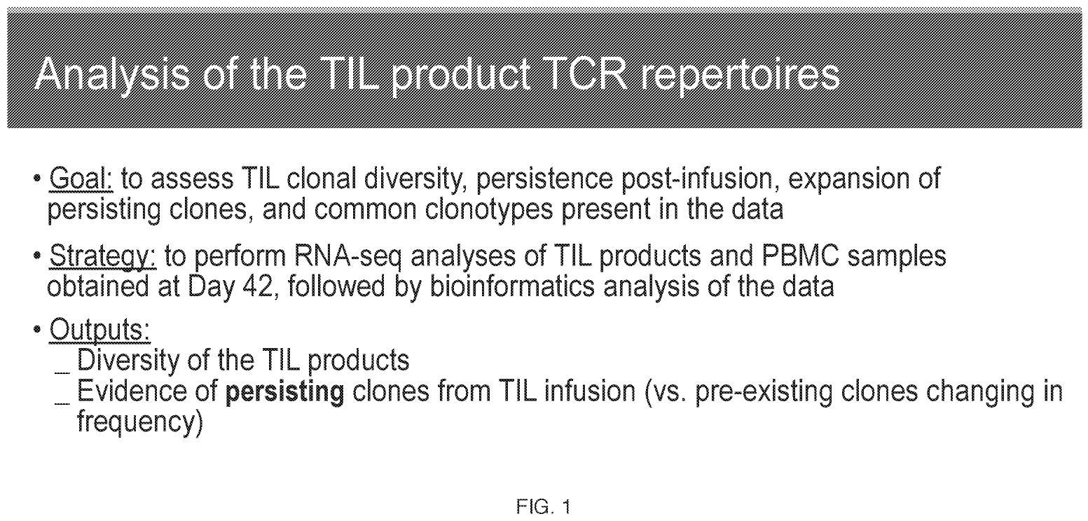

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

The term “immunorepertoire” means the set of distinct CDR3 sequences detected in the lymphocytes of an individual or individuals, as applicable.

Clonotypes, also known as “clonal types,” of an immunorepertoire are determined by the rearrangement of Variable (V), Diverse (D) and Joining (J) gene segments through somatic recombination in the early stages of immunoglobulin (Ig) and T cell receptor (TCR) production of the immune system. The V(D)J rearrangement can be amplified and detected from T cell receptor alpha, beta, gamma, and delta chains, as well as from immunoglobulin heavy chain (IgH) and light chains (IgK, IgL). Cells may be obtained from a patient by obtaining peripheral blood, lymphoid tissue, cancer tissue, or tissue or fluids from other organs and/or organ systems, for example. Techniques for obtaining these samples, such as blood samples, are known to those of skill in the art. Cell counts may be extrapolated from the number of sequences detected by PCR amplification and sequencing.

The CDR3 region, comprising about 30-90 nucleotides, encompasses the junction of the recombined variable (V), diversity (D) and joining (J) segments of the gene. It encodes the binding specificity of the receptor and is useful as a sequence tag to identify unique V(D)J rearrangements.

Wang et al. disclosed that PCR may be used to obtain quantitative or semi-quantitative assessments of the numbers of target molecules in a specimen (Wang, M. et al, “Quantitation of mRNA by the polymerase chain reaction,” Proc. Nat'l. Acad. Sci. 86:9717-9721 (1989)). Particularly effective methods for achieving quantitative amplification have been described previously by the inventor. One such method is known as arm-PCR, which is described in United States Patent Application Publication Number 2009/0253183A1.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “ex vivo” refers to an event which involves treating or performing a procedure on a cell, tissue, and/or organ which has been removed from a subject's body. Aptly, the cell, tissue and/or organ may be returned to the subject's body in a method of surgery or treatment.

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs include both primary and secondary TILs. “Primary TILs” are those that are obtained from patient tissue samples as outlined herein (sometimes referred to as “freshly obtained” or “freshly isolated”), and “secondary TILs” are any TIL cell populations that have been expanded or proliferated as discussed herein, including, but not limited to bulk TILs and expanded TILs (“REP TILs” or “post-REP TILs”). TIL cell populations can include genetically modified TILs.

By “population of cells” (including TILs) herein is meant a number of cells that share common traits. In general, populations generally range from 1×10⁶ to 1×10¹⁰ in number, with different TIL populations comprising different numbers. For example, initial growth of primary TILs in the presence of IL-2 results in a population of bulk TILs of roughly 1×10⁸ cells. REP expansion is generally done to provide populations of 1.5×10⁹ to 1.5×10¹⁰ cells for infusion. In some embodiments, REP expansion is done to provide populations of 2.3×10¹⁰ 13.7×10¹⁰.

By “cryopreserved TILs” herein is meant that TILs, either primary, bulk, or expanded (REP TILs), are treated and stored in the range of about −150° C. to −60° C. General methods for cryopreservation are also described elsewhere herein, including in the Examples. For clarity, “cryopreserved TILs” are distinguishable from frozen tissue samples which may be used as a source of primary TILs.

By “thawed cryopreserved TILs” herein is meant a population of TILs that was previously cryopreserved and then treated to return to room temperature or higher, including but not limited to cell culture temperatures or temperatures wherein TILs may be administered to a patient.

TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally, and alternatively, TILs can be functionally defined by their ability to infiltrate solid tumors upon reintroduction into a patient.

The term “central memory T cell” refers to a subset of T cells that in the human are CD45R0+ and constitutively express CCR7 (CCR7^{hi}) and CD62L (CD62^{hi}). The surface phenotype of central memory T cells also includes TCR, CD3, CD127 (IL-7R), and IL-15R. Transcription factors for central memory T cells include BCL-6, BCL-6B, MBD2, and BMI1. Central memory T cells primarily secrete IL-2 and CD40L as effector molecules after TCR triggering. Central memory T cells are predominant in the CD4 compartment in blood, and in the human are proportionally enriched in lymph nodes and tonsils.

The term “effector memory T cell” refers to a subset of human or mammalian T cells that, like central memory T cells, are CD45R0+, but have lost the constitutive expression of CCR7 (CCR7^{lo}) and are heterogeneous or low for CD62L expression (CD62L^{lo}). The surface phenotype of central memory T cells also includes TCR, CD3, CD127 (IL-7R), and IL-15R. Transcription factors for central memory T cells include BLIMP1. Effector memory T cells rapidly secrete high levels of inflammatory cytokines following antigenic stimulation, including interferon-γ, IL-4, and IL-5. Effector memory T cells are predominant in the CD8 compartment in blood, and in the human are proportionally enriched in the lung, liver, and gut. CD8+ effector memory T cells carry large amounts of perforin.

The terms “peripheral blood mononuclear cells” and “PBMCs” refer to a peripheral blood cell having a round nucleus, including lymphocytes (T cells, B cells, NK cells) and monocytes. When used as antigen-presenting cells (PBMCs are a type of antigen-presenting cell), the peripheral blood mononuclear cells are irradiated allogeneic peripheral blood mononuclear cells.

The terms “peripheral blood lymphocytes” and “PBLs” refer to T cells expanded from peripheral blood. In some embodiments, PBLs are separated from whole blood or apheresis product from a donor. In some embodiments, PBLs are separated from whole blood or apheresis product from a donor by positive or negative selection of a T cell phenotype, such as the T cell phenotype of CD3+CD45+.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The term “clinically effective” in the context of a population of TILs or other T cells as used herein encompasses a clinically detectable therapeutic effect and/or a prophylactic effect. Non-limiting examples of a clinically detectable therapeutic effect include a reduction in solid tumor mass; and, patient reported reduction in symptoms, for example, pain or discomfort. A prophylactic effect includes delaying or eliminating the appearance of a disease or condition, delaying or eliminating the onset of symptoms of a disease or condition, slowing, halting, or reversing the progression of a disease or condition, or any combination thereof.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

Compounds of the invention also include antibodies. The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (Clq) of the classical complement system.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird et al., Science 1988, 242, 423-426; and Huston et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes. In mammals, there are five antibody isotypes: IgA, IgD, IgG, IgM and IgE. In humans, there are four subclasses of the IgG isotype: IgG1, IgG2, IgG3 and IgG4, and two subclasses of the IgA isotype: IgA1 and IgA2.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to a therapeutic moiety, such as a bacterial toxin, a cytotoxic drug or a radionuclide-containing toxin. Toxic moieties can be conjugated to antibodies of the invention using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 1986, 321, 522-525; Riechmann et al., Nature 1988, 332, 323-329; and Presta, Curr. Op. Struct. Biol. 1992, 2, 593-596.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., European Patent No. EP 404,097, International Patent Publication No. WO 93/11161; and Bolliger et al., Proc. Natl. Acad. Sci. USA 1993, 90, 6444-6448.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki, et al. Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields, et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana, et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino, et al., Biochem. 1975, 14, 5516-5523.

“Pegylation” refers to a modified antibody, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g., serum) half-life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. The antibody to be pegylated may be an aglycosylated antibody. Methods for pegylation are known in the art and can be applied to the antibodies of the invention, as described for example in European Patent Nos. EP 0154316 and EP 0401384.

The term “biosimilar” means a biological product that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference anti-CD20 monoclonal antibody is rituximab, an anti-CD20 biosimilar monoclonal antibody approved by drug regulatory authorities with reference to rituximab is a “biosimilar to” rituximab or is a “biosimilar thereof” of rituximab. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

The terms “sequence identity,” “percent identity,” and “sequence percent identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. ClustalW and ClustalX may be used to produce alignments, Larkin et al., Bioinformatics 23:2947-2948 (2007); Gouj on et al., Nucleic Acids Research, 38 Suppl:W 695-9 (2010); and, McWilliam et al., Nucleic Acids Research 41 (Web Server issue):W 597-600 (2013). One skilled in the art can determine appropriate parameters for maximal alignment by particular alignment software. In certain embodiments, the default parameters of the alignment software are used.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

The term “solid tumor” refers to an abnormal mass of tissue that usually does not contain cysts or liquid areas. Solid tumors may be benign or malignant. The term “solid tumor cancer refers to malignant, neoplastic, or cancerous solid tumors. Solid tumor cancers include, but are not limited to, sarcomas, carcinomas, and lymphomas, such as cancers of the lung, breast, triple negative breast cancer, prostate, colon, rectum, and bladder. In some embodiments, the cancer is selected from cervical cancer, head and neck cancer, glioblastoma, ovarian cancer, sarcoma, pancreatic cancer, bladder cancer, breast cancer, triple negative breast cancer, and non-small cell lung carcinoma. The tissue structure of solid tumors includes interdependent tissue compartments including the parenchyma (cancer cells) and the supporting stromal cells in which the cancer cells are dispersed and which may provide a supporting microenvironment.

The term “hematological malignancy” refers to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, and non-Hodgkin's lymphomas. The term “B cell hematological malignancy” refers to hematological malignancies that affect B cells.

The term “comparable” or “comparability” as used herein can refer to a quality that two or more samples are similar. In certain embodiments, two or more samples that are comparable have the same or about the same therapeutic efficacy. In certain embodiments, two or more samples that are “comparable” have the same or about the same CDR3 clonal diversity. Use of the term “about” is an approximation within experimental variability (or within statistical experimental error). The variation is typically from 0% to 15%, preferably from 0% to 10%, more preferably from 0% to 5% of the stated number or numerical range.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

### TIL Expansion Methods

Various methods for expanding tumor infiltrating lymphocytes to produce a therapeutic population of TILs are methods known to the art. The methods below are nonlimiting. It is understood that the systems and methods disclosed herein are compatible with all methods of TIL, marrow infiltrating lymphocyte (MIL), and PBL expansion, culture, and manufacturing.

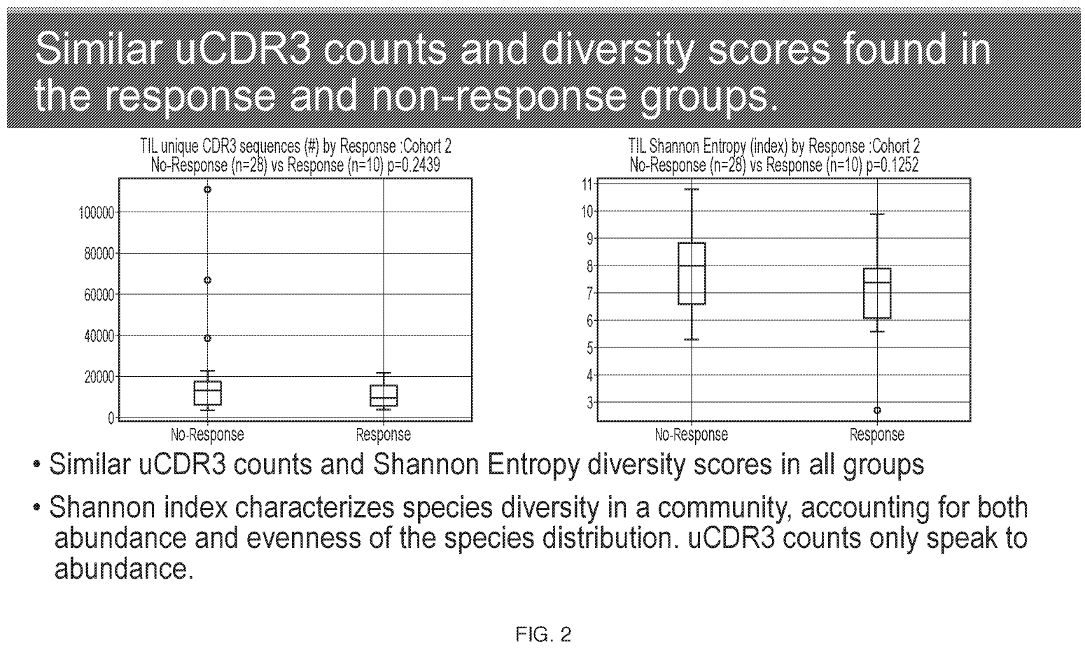

An exemplary TIL process known as Process 2A is disclosed in U.S. Pat. No. 10,130,659, which is incorporated by reference in its entirety, with particular attention drawn to FIG. 2 comparing Process 1C to Process 2A, and the various embodiments of Process 2A disclosed in U.S. Pat. No. 10,130,659. Exemplary Process 2A TIL manufacturing methods are disclosed in U.S. Patent Application Publication No. US 2018/0282694A1, which is incorporated by reference in its entirety. Exemplary Process 2A TIL manufacturing methods are also disclosed in U.S. Patent Application Publication No. US2018/0207201A1, which is incorporated by reference in its entirety.

Other methods for expanding TILs are discussed in the following: Dudley, et al., Science 2002, 298, 850-54; Dudley, et al., J. Clin. Oncol. 2005, 23, 2346-57; Dudley, et al., J. Clin. Oncol. 2008, 26, 5233-39; Riddell, et al., Science 1992, 257, 238-41; Dudley, et al., J. Immunother. 2003, 26, 332-42. Rohann et al., Journal for ImmunoTherapy of Cancer 2018, 6:102-118, discuss TIL manufacturing generally and clinical uses of therapeutic TIL populations.

TILs may be expanded according to any of the above methods to produce a population of cells suitable for use according to the methods and systems disclosed herein.

### CDR3 Clonotype Methods

### Nucleic Acid Amplification Methods

Whole cell nucleic acid may be isolated from a population of lymphocytes and amplified using various methods, including dimer avoided multiplex polymerase chain reaction (dam-PCR), e.g. as disclosed in International Patent Publication No. WO/2018/165593, the international publication of PCT/US2018/021816, which is an embodiment of multiplex PCR. Han et al., and/or U.S. Pat. No. 9,938,578, disclose multiplex methods for analyzing mixed nucleotide samples comprising using polynucleotide amplification to produce amplified products wherein one or more target sequences are tagged with a non-interfering, non-canceling target-specific polynucleotide identification tag, and pyrosequencing the amplified products through the non-canceling target-specific polynucleotide identification tag sequence to detect the presence of one or more specific polynucleotide identification tags. The presence of a specific polynucleotide identification tag is correlated with the presence of a specific target sequence.

Other useful methods include, but are not limited to, those disclosed by Han in U.S. Patent Application Publication No. US2017/0088895A1, which is incorporated by reference in its entirety, with particular focus on methods using primers directed to CDR3 sequences to amplify and assemble a representative collection of CDR3 diversity present in the source sample. Such methods include methods for determining the diversity of a patient's immunorepertoire by comparing the CDR3 sequences in a patient sample to the most commonly-shared CDR3 sequences (i.e., the pCDR3) of an index group of individuals. This percentage of pCDR3 in a patient's sample is referred to as the “normality index” and may serve as a diagnostic indicator of the immunorepertoire diversity in such patient. The present disclosure further includes a method for determining such a subset of highly-shared pCDR3. In one aspect of the present disclosure, the immunorepertoire of a patient is considered normal if the patient's normality index meets or exceeds a minimum percentage, whereas the immunorepertoire of the patient is considered abnormal of the patient's normality index is below such minimum percentage.

The CDR3 expressed by individuals exhibits tremendous diversity, with up to 10¹⁵ unique CDR3 possible. As such, the present disclosure uses CDR3 as a basis for immune system diversity. Han (U.S. Patent Application Publication No. US2017/0088895A1) discloses, based on a sampling of 75 million CDR3, that approximately 81% of randomly-selected CDR3 are unique to a given individual and are not shared among multiple individuals. Han (U.S. Patent Application Publication No. US2017/0088895A1) provides a method for determining a pool of highly-shared CDR3, thereby enabling a standard index of shared CDR3 by which the diversity of an individual's immunorepertoire may be identified. Furthermore, a single subject's immunorepertoire at two different points in time may be determined, and then, using the methods disclosed herein, compared as detailed below.

Lymphocytes, e.g., T cells and/or T cell subsets may be isolated and/or sorted using techniques known in the art, including, but not limited to, apheresis, separation of PBMC using Ficoll-paque gradients, FACS, and magnetic bead separation methods.

To determine the immunorepertoire, comprising the set of distinct CDR3 sequences, the following exemplary approach may be used: (a) amplifying polynucleotides from a population of white blood cells from a patient in a reaction mix comprising target-specific nested primers to produce a set of first amplicons, at least a portion of the target-specific nested primers comprising additional nucleotides which, during amplification, serve as a template for incorporating into the first amplicons a binding site for at least one common primer; (b) transferring a portion of the first reaction mix containing the first amplicons to a second reaction mix comprising at least one common primer; (c) amplifying, using the at least one common primer, the first amplicons to produce a set of second amplicons; (d) sequencing the second amplicons to identify CDR3 sequences in the subpopulation of white blood cells, and (e) using the identified CDR3 sequences to quantify the percentage of pCDR3 represented by the sample to provide a normality index; and (f) identifying whether the normality index is normal or abnormal, wherein a normal state is characterized by the presence of a minimum percentage of pCDR3 and an abnormal state is characterized by the absence of a minimum percentage of pCDR3. Such methods are known to the art and described by Han in U.S. Patent Application Publication No. US2017/0088895A1.

Other useful methods are disclosed by Han in U.S. Pat. No. 7,999,092, which is incorporated by reference in its entirety, with attention drawn to the arm-PCR and arm-RT-PCR methods. In summary, Han (U.S. Pat. No. 7,999,092) describes a method for amplifying nucleic acids to enable detection of those nucleic acids, the method comprising the steps of amplifying one or more target nucleic acids using high concentration, target-specific primers in a first amplification reaction, thereby producing at least one nucleic acid amplicon containing at least one common primer binding site; rescuing the at least one nucleic acid amplicon; and amplifying the at least one nucleic acid amplicon in a second amplification reaction utilizing common primers which bind to the at least one common primer binding site. One aspect of the invention utilizes nested target-specific primers. Target nucleic acids may comprise DNA and/or RNA, and may comprise human genomic DNA and/or RNA. Amplification may be performed by polymerase chain reaction (PCR) and/or RT-PCR. The source of the target nucleic acids may be from one or more clinical, environmental, or food samples and the method may be used in a wide variety of ways, including, for example, clinical diagnosis, environmental sampling, plant testing, food safety analysis, detection of genetic disorders, and/or detection of disease conditions. The method may be used for human and/or veterinary medical diagnoses.

Another exemplary approach employing various Han disclosures described above and incorporated herein by reference to determine the CDR3 repertoire of a population of lymphocytes, include arm-PCR and/or arm-RT-PCR, in a multi-step reaction to quantitatively amplify an immune repertoire. During the first round of PCR, nested gene specific primers targeting each of the V and C genes are used. The forward primers Fo (forward-out) and Fi (forward-in) are located in the V genes. The reverse primers, Ro (reverse-out) and Ri (reverse-in), are located in each of the C genes. The Fi and Ri primers also include sequencing adaptors B and A, respectively, for the Roche 454 platforms (454 and the GS Junior). For the Ri primers, there are also barcodes in between the sequencing primer A and the C gene specific primers. As a result, sequencing is restricted to single-end reads from primer A only. The second round of PCR is carried out using communal (sequencing) primers B and A. After gel purification, the resulting product is ready for high throughput sequencing with the Roche 454 platforms. No additional enzymatic steps are required. The first round of PCR introduces barcodes and sequencing primers into the PCR products. The exponential phase of the amplification is achieved by the communal primers in the second round of PCR; therefore, the entire repertoire is amplified evenly and semi-quantitatively, without introducing additional amplification bias. Genomic DNA, RNA, mRNA, mixed cellular nucleic acid samples, and/or cDNA library, are suitable starting points for such immune repertoire amplification methods.

Alternative approaches to measuring and determining clonotype profiles are disclosed in U.S. Pat. No. 10,155,992, which is incorporated by reference in its entirety, with particular attention drawn the methods for amplifying rearranged T-cell receptor CDR3-encoding region DNA molecules. Faham and Willis teach detailed methods for developing T-cell receptor (TCR) CDR3 clonontypes from amplified DNA. Such methods are complimentary to RNA focused methods and are used advantageously by the present invention. In one aspect, the present invention provides methods of identifying clinically effective TIL populations utilizing both mRNA clonotype data and DNA.

Because the recombinations are present in the DNA of each individual's adaptive immunity cells as well as their associated RNA transcripts, either RNA (including mRNA) or DNA can be sequenced in the methods of the provided invention. A recombined sequence from a T cell or B cell encoding a T cell receptor or immunoglobulin molecule, or a portion thereof, is referred to as a clonotype. The DNA or RNA (including mRNA) can correspond to sequences from T-cell receptor (TCR) genes or immunoglobulin (Ig) genes that encode antibodies. For example, the DNA and RNA can correspond to sequences encoding α, β, γ, or δ chains of a TCR. In a majority of T cells, the TCR is a heterodimer consisting of an α-chain and β-chain. The TCRα chain is generated by VJ recombination, and the β chain receptor is generated by V(D)J recombination. For the TCRβ chain, in humans there are 48 V segments, 2 D segments, and 13 J segments. Several bases may be deleted and others added (called N and P nucleotides) at each of the two junctions. In a minority of T cells, the TCRs consist of γ and δ delta chains. The TCR γ chain is generated by VJ recombination, and the TCR δ chain is generated by V(D)J recombination (Kenneth Murphy et al., Janeway's Immunology 9th edition, Garland Science, 2016, ISBN-13: 978-0815345503).

A feature of the methods taught in U.S. Pat. No. 10,559,992 are that clonotype expression may be measured at the cellular level. For example, clonotypes may be used to count lymphocytes by measuring clonotypes derived from genomic DNA and the same clonotypes derived from RNA, whereby cell-based expression of clonotypes may be determined. A method for simultaneously measuring lymphocyte numbers and clonotype expression levels in a sample may comprise the steps of: (a) obtaining front an individual a sample comprising T cells and/or B cells; (b) sequencing spatially isolated individual molecules derived from genomic DNA of said cells, such spatially isolated individual molecules comprising a number of clonotypes corresponding to a number of lymphocytes in the sample; (c) sequencing spatially isolated individual molecules derived from RNA of said cells, such spatially isolated individual molecules comprising numbers of clonotypes corresponding to expression levels thereof in the lymphocytes of the sample; and (d) determining clonotype expression levels in lymphocytes of the sample by comparing for each clonotype the number determined from isolated individual molecules derived from genomic DNA of said cells and the number determined from isolated individual molecules derived from RNA of said cells.

Guidance for carrying out multiplex PCRs of such immune molecules is found in the following references, which are incorporated by reference: Morley, U.S. Pat. No. 5,296,351; Gorski, U.S. Pat. No. 5,837,447; Dau, U.S. Pat. No. 6,087,096; Von Dongen et al., U.S. Patent Publication No. 2006/0234234; European Patent EP 1544308B1, all of which are incorporated herein by reference.

Other means of amplifying nucleic acid that can be used in the methods of the provided invention include, for example, reverse transcription-PCR, real-time PCR, quantitative real-time PCR, digital PCR (dPCR), digital emulsion PCR (dcPCR), clonal PCR, amplified fragment length polymorphism PCR (AFLP PCR), allele specific PCR, assembly PCR, asymmetric PCR (in which a great excess of primers for a chosen strand is used), colony PCR, helicase-dependent amplification (HDA), Hot Start PCR, inverse PCR (IPCR), in situ PCR, long PCR (extension of DNA greater than about 5 kilobases), multiplex PCR, nested PCR (uses more than one pair of primers), single-cell PCR, touchdown PCR, loop-mediated isothermal PCR (LAMP), and nucleic acid sequence based amplification (NASBA). Other amplification schemes include: Ligase Chain Reaction, Branch DNA Amplification, Rolling Circle Amplification, Circle to Circle Amplification, SPIA amplification, Target Amplification by Capture and Ligation (TACL) amplification, and RACE amplification.

The information in RNA (including mRNA) in a sample can be converted to cDNA by using reverse transcription. PolyA primers, random primers, and/or gene specific primers can be used in reverse transcription reactions in accordance with conventional protocols.

Furthermore, for example, by amplification of DNA from the genome (or amplification of nucleic acid in the form of cDNA by reverse transcribing RNA or mRNA), the individual nucleic acid molecules can be isolated, optionally re-amplified, and then sequenced individually. Exemplary amplification protocols may be formed in van Dongen et al., Leukemia, 17: 2257-2317 (2003) or van Dongen et al., U.S. Pat. No. 8,859,748, which is incorporated by reference in its entirety. Briefly, an exemplary protocol is as follows: Reaction buffer: ABI Buffer II or ABI Gold Buffer (Life Technologies, San Diego, Calif.); 50 μL final reaction volume; 100 ng sample DNA; 10 pmol of each primer (subject to adjustments to balance amplification as described below); dNTPs at 200 μM final concentration; MgCl₂ at 1.5 mM final concentration (subject to optimization depending on target sequences and polymerase); Taq polymerase (1-2 U/tube); cycling conditions: preactivation 7 min at 95° C.; annealing at 60° C.; cycling times: 30 second denaturation; 30 second annealing; 30 second extension.

Methods for isolation of nucleic acids from a pool include, but are not limited to, spatial separation of the molecules in two dimensions on a solid substrate (e.g., glass slide), spatial separation of the molecules in three dimensions in a solution within micelles (such as can be achieved using oil emulsions with or without immobilizing the molecules on a solid surface such as beads), or using microreaction chambers in, for example, microfluidic or nano-fluidic chips. Dilution can be used to ensure that on average a single molecule is present in a given volume, spatial region, bead, or reaction chamber. Guidance for such methods of isolating individual nucleic acid molecules is found in the following references: Green and Sambrook, Molecular Cloning: A Laboratory Manual (Cold Spring Harbor Laboratory Press, 2012, ISBN: 978-936113-42-2); Shendure et al., Science, 309: 1728-1732 (including supplemental material) (2005); U.S. Pat. No. 6,300,070; Bentley et al., Nature 456: 53-59 (including supplemental material) (2008); U.S. Pat. No. 7,323,305; Matsubara et al., Biosensors & Bioelectronics, 20:1482-1490 (2005); U.S. Pat. No. 6,753,147, all of which are incorporated herein by reference.

Furthermore, any high-throughput technique for sequencing nucleic acids can be used in the methods of the invention. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of the separated molecules has more recently been demonstrated by sequential or single extension reactions using polymerases or ligases as well as by single or sequential differential hybridizations with libraries of probes. These reactions have been performed on many clonal sequences in parallel including demonstrations in current commercial applications of over 100 million sequences in parallel.

These sequencing approaches can thus be used to study the repertoire of T-cell receptor (TCR) and/or B-cell receptor (BCR). In one aspect of the invention, high-throughput methods of sequencing are employed that comprise a step of spatially isolating individual molecules on a solid surface where they are sequenced in parallel. Such solid surfaces may include nonporous surfaces (such as in Solexa sequencing, e.g., Bentley et al., Nature 456: 53-59 (2008) or Complete Genomic sequencing, e.g. Drmanac et al., Science 327: 78-81 (2010)), arrays of wells, which may include bead- or particle-bound templates (such as with 454, e.g. Margulies et al., Nature 437:376-380 (2005); Ion Torrent sequencing, U.S. Pat. No. 8,574,835), micromachined membranes (such as with SMRT sequencing, e.g. Eid et al., Science 323:133-138 (2009)), or bead arrays (as with SOLiD sequencing or polony sequencing, e.g. Kim et al., Science 316:1481-1414 (2007)). In various clonotype amplifications, such nucleic acids are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; or EP Patent No. EP0972081B1; which are incorporated by reference.

In one embodiment, individual molecules disposed and amplified on a solid surface form clusters in a density of at least 10⁵ clusters per cm²; or in a density of at least 5×10⁵ per cm²; or in a density of at least 10⁶ clusters per cm². In one embodiment, sequencing chemistries are employed having relatively high error rates. In such embodiments, the average quality scores produced by such chemistries are monotonically declining functions of sequence read lengths. In one embodiment, such decline corresponds to 0.5 percent of sequence reads having at least one error in positions 1-75; 1 percent of sequence reads having at least one error in positions 76-100; and 2 percent of sequence reads having at least one error in positions 101-125.

Additional methods useful for determining immune cell clonotypes, and in particular, identifying the T-cell receptor (TCR) complementarity determining region 3 (CDR3)-encoding nucleic acid sequence clones constituting the TCR CDR3 clonal diversity of a therapeutic population of TILs are disclosed in U.S. Pat. Nos. 10,150,996; 10,077,478; 10,077,473; 10,066,265; 9,824,179; 9,528,160; and 9,499,865, each of which is incorporated by reference in its entirety, with particular focus on amplification methods.

Similarly, useful methods for determining immune cell clonotypes, and in particular, identifying the T-cell receptor (TCR) complementarity determining region 3 (CDR3)-encoding nucleic acid sequence clones constituting the TCR CDR3 clonal diversity of a population of PBMCs are disclosed in U.S. Pat. Nos. 10,150,996; 10,077,478; 10,077,473; 10,066,265; 9,824,179; 9,528,160; and 9,499,865. Such methods may be comparable or complementary to mRNA-based methods described herein.

### Methods for Identifying Clinically Effective Populations of TILs

In one aspect, the invention is directed to a method for identifying a clinically effective population of tumor infiltrating lymphocytes (TILs), the method comprising: identifying a clinically effective population of tumor infiltrating lymphocytes (TILs) in a therapeutic population of TILs administered to a subject, the method comprising:

(i) identifying the T cell receptor (TCR) complementarity determining region 3 (CDR3)-encoding nucleic acid sequence clones constituting the TCR CDR3 clonal diversity of a therapeutic population of TILs;

(ii) identifying the TCR CDR3-encoding nucleic acid sequence clones constituting the TCR CD3 clonal diversity of a first population of peripheral blood mononuclear cells (PBMCs), wherein the first population of PBMCs is isolated from a subject at least 14-days after the therapeutic population of step (i) is administered to said subject;

(iii) for each unique TCR CDR3-encoding nucleic acid sequence clone identified in step (ii), determining the frequency of such unique TCR CDR3 clone in each of the therapeutic population of TILs and the first population of PBMCs;

(iv) sorting the unique TCR CDR3-encoding nucleic acid sequence clones identified in step (ii) from highest frequency to lowest frequency for each of the therapeutic population of TILs and the first population of PBMCs; and,

(v) selecting the ten highest frequency unique TCR CDR3-encoding nucleic acid sequence clones from the first population of PBMCs sorted in step (iv), wherein the TILs expressing such clones in the therapeutic population of TILs constitute a clinically effective population of TILs, thereby identifying the clinically effective population of TILs.

In various embodiments, identifying the T cell receptor (TCR) complementarity determining region 3 (CDR3)-encoding nucleic acid sequence clones constituting the TCR CDR3 clonal diversity is performed using methods known to one skilled in the art, including, but not limited to the methods described herein. In various embodiments, identifying the TCR CDR3-encoding nucleic acid sequence clones constituting the TCR CD3 clonal diversity of a first population of peripheral blood mononuclear cells (PBMCs), is performed using methods known to one skilled in the art, including, but not limited to the methods described herein.

In another aspect, the invention is directed to a method for identifying a clinically effective population of tumor infiltrating lymphocytes (TILs), the method further comprising: the steps of (a) identifying the TCR CDR3-encoding nucleic acid sequence clones constituting the TCR CDR3 clonal diversity of a second population of PBMCs isolated from the subject prior to administration of the therapeutic population of TILs to the subject; and (b) determining the frequency of each unique TCR CDR3-encoding nucleic acid sequence clone identified in step (a).

In another aspect, the invention is directed to a method for identifying a clinically effective population of tumor infiltrating lymphocytes (TILs), wherein the TCR CDR3-encoding nucleic acid sequence clones constituting the clonal diversity of the second population of PBMCs are different from the TCR CDR3-encoding nucleic acid sequence clones constituting the clonal diversity of the first population of PBMCs isolated from the subject post administration of the therapeutic population of TILs.

In another aspect, the invention is directed to a method for identifying a clinically effective population of tumor infiltrating lymphocytes (TILs), wherein the frequency of unique TCR CDR3-encoding nucleic acid sequence clones identified in at least one population is determined by DNA sequencing.

In some aspects, the invention is directed to a method for identifying a clinically effective population of tumor infiltrating lymphocytes (TILs), wherein the frequency of unique TCR CDR3-encoding nucleic acid sequence clones identified in at least one population is determined by RNA sequencing. In some aspects, the invention is directed to a method for identifying a clinically effective population of tumor infiltrating lymphocytes (TILs), wherein the frequency of unique TCR CDR3-encoding nucleic acid sequence clones identified in the therapeutic population of TILs and the frequency of unique TCR CD3-encoding nucleic acid sequence clones identified in the first population of PBMCs are determined by both DNA and RNA sequencing.

Another aspect of the present invention provides for a method for identifying a clinically effective population of tumor infiltrating lymphocytes (TILs), wherein the frequency of unique TCR CDR3-encoding nucleic acid sequence clones identified in the first population of PBMCs as determined by RNA sequencing is compared to the frequency of unique TCR CDR3-encoding nucleic acid sequence clones identified in the first population of PBMCs as determined by DNA sequencing, wherein the frequency of such unique clones as determined by RNA sequencing is indicative of a clinically effective population of TILs as compared to the frequency of such unique clones as determined by DNA sequencing.

Another aspect of the present invention provides for a method for identifying a clinically effective population of tumor infiltrating lymphocytes (TILs), wherein the frequency of such unique clones is greater as determined by RNA sequencing. Another aspect of the present invention provides for a method for identifying a clinically effective population of tumor infiltrating lymphocytes (TILs), wherein the frequency of such unique clones is greater as determined by DNA sequencing.

Yet another aspect of the present invention provides for a method for identifying a clinically effective population of tumor infiltrating lymphocytes (TILs), wherein the frequency of such unique clones as determined by RNA sequencing correlates to a population of TILs with enhanced therapeutic efficacy. Other aspects of the present invention provide for a method for identifying a clinically effective population of tumor infiltrating lymphocytes (TILs), wherein the frequency of such unique clones as determined by DNA sequencing does not correlate to a population of TILs with enhanced therapeutic efficacy. Other aspects of the present invention provide for a method for identifying a clinically effective population of tumor infiltrating lymphocytes (TILs), wherein the frequency of such unique clones as determined by RNA sequencing correlates to a population of TILs with enhanced therapeutic efficacy and the frequency of such unique clones as determined by DNA sequencing does not correlate to a population of TILs with enhanced therapeutic efficacy.

In another aspect, the invention is directed to a method for identifying a clinically effective population of tumor infiltrating lymphocytes (TILs), wherein the TCR CDR3-encoding nucleic acid sequence clones are mRNA clones, which are identified by RNA sequencing.

The methods of the present disclosure may be performed at various times after the administration of a therapeutic population of cells. Methods for identifying a clinically effective population of TILs may be performed about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, and/or about 60 days after the administration of a therapeutic population of TILs.

In some embodiments, methods for identifying a clinically effective population of TILs are performed about 20 days, about 25 days, about 30 days, about 35 days, about 40 days, about 42 days, about 45 days, about 50 days, about 55 days, and/or about 60 days after administration of a therapeutic population of TILs. In other embodiments, methods for identifying a clinically effective population of TILs may be performed about 20 days, about 25 days, about 30 days, about 35 days, about 40 days, about 42 days, about 45 days, about 50 days, about 55 days, about 60 days, about 90 days, about 120 days, about 180 days, about 1 year, about 2 years, about 3 years, about 4 years, and/or about 5 years after the administration of a therapeutic population of TILs.

In some embodiments, methods for identifying a clinically effective population of TILs are capable of detecting mRNA about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, and/or about 60 days after administration of a therapeutic population of TILs.

In some embodiments, methods for identifying a clinically effective population of TILs are capable of detecting mRNA about 20 days, about 25 days, about 30 days, about 35 days, about 40 days, about 42 days, about 45 days, about 50 days, about 55 days, and/or about 60 days after administration of a therapeutic population of TILs. In other embodiments, methods for identifying a clinically effective population of TILs are capable of detecting mRNA about 20 days, about 25 days, about 30 days, about 35 days, about 40 days, about 42 days, about 45 days, about 50 days, about 55 days, about 60 days, about 90 days, about 120 days, about 180 days, about 1 year, about 2 years, about 3 years, about 4 years, and/or about 5 years after the administration of a therapeutic population of TILs.

Another aspect of the invention provides for a method for enhancing a subject's T-cell repertoire, the method comprising: (i) identifying a clinically effective population of tumor infiltrating lymphocytes according to the present disclosure; and, (ii) selecting and expanding the population identified in step (i) to produce a second a clinically effective therapeutic population of TILs. In other aspects, the invention further provides for administering the expanded cells produced in step (ii), thereby enhancing the subject's T-cell repertoire.

In some embodiments, nucleic acids are analyzed from a sample of a subset of cells. A method to separate cells, for example by using a cell surface marker, can be employed. For example, cells can be isolated by cell sorting flow-cytometry, flow-sorting, fluorescent activated cell sorting (FACS), bead based separation such as magnetic cell sorting (MACS; e.g., using antibody coated magnetic particles), size-based separation (e.g., a sieve, or a filter), sorting in a microfluidics device, antibody-based separation, sedimentation, affinity adsorption, affinity extraction, or density gradient centrifugation. Sorting can be based on cell size, morphology, or intracellular or extracellular markers. Methods for isolating or sorting tumor cells are described, for example, in Nagrath et al. Nature 450:1235-1239 (2007); U.S. Pat. Nos. 6,008,002, 7,232,653 and 7,332,288; PCT Publication No. WO2008157220A1; and U.S. Patent Application Publication Nos. US2008/0138805A1 and US2009/0186065; or Rosenberg et al. Cytometry 49:150-158 (2002), each of which is herein incorporated by reference in their entireties.

### Methods for Determining Persistence

In one aspect, the invention is directed to a method for determining the persistence and activity of T cell receptor (TCR) complementarity determining region 3 (CDR3)-encoding nucleic acid sequence clones in tumor infiltrating lymphocytes (TILs) in a therapeutic population of TILs administered to a subject, the method comprising:

(a) identifying the TCR CDR3-encoding nucleic acid clones constituting the TCR CDR3 clonal diversity of a therapeutic population of TILs;

(b) identifying the TCR CDR3-encoding nucleic acid sequence clones constituting the TCR CDR3 clonal diversity of a first population of peripheral blood mononuclear cells (PBMCs), wherein the first population of PBMCs is isolated from a subject at least 14-days after the therapeutic population of step (a) is administered to said subject;

(c) for each unique TCR CDR3-encoding nucleic acid sequence clone identified in step (b), determining the frequency of such unique TCR CDR3-encoding nucleic acid sequence clone in each of the therapeutic population of TILs and the first population of PBMCs; and,

(d) for each unique TCR CDR3-encoding nucleic acid sequence clone identified in step (b), comparing the frequency of the TCR CDR3-encoding nucleic acid sequence clone in the first population of PBMCs to the frequency of the TCR CDR3-encoding nucleic acid sequence clone in the therapeutic population of TILs to determine the persistence and activity of TCR CDR3-encoding nucleic acid sequence clones in the therapeutic TIL population administered to the subject.

In another aspect, the invention is directed to a method for determining the persistence and activity of T cell receptor (TCR) complementarity determining region 3 (CDR3)-encoding nucleic acid sequence clones in tumor infiltrating lymphocytes (TILs) in a therapeutic population of TILs administered to a subject, wherein the frequency of unique TCR CDR3-encoding nucleic acid sequence clones identified in the therapeutic population of TILs and the frequency of unique TCR CDR3-encoding nucleic acid sequence clones identified in the first population of PBMCs are determined by both DNA and RNA (including mRNA) sequencing.

In another aspect, the invention is directed to a method for determining the persistence and activity of T cell receptor (TCR) complementarity determining region 3 (CDR3)-encoding nucleic acid sequence clones in tumor infiltrating lymphocytes (TILs) in a therapeutic population of TILs administered to a subject, wherein the frequency of unique TCR CDR3-encoding nucleic acid sequence clones identified in the first population of PBMCs as determined by RNA (including mRNA) sequencing is compared to the frequency of unique TCR CDR3-encoding nucleic acid sequence clones identified in the first population of PBMCs as determined by DNA sequencing, wherein the frequency of such unique clones as determined by RNA (including mRNA) sequencing compared to the frequency of such unique clones as determined by DNA sequencing is indicative of the persistence and activity of such clones.

In another aspect, the invention is directed to a method for determining the persistence and activity of T cell receptor (TCR) complementarity determining region 3 (CDR3)-encoding nucleic acid sequence clones in tumor infiltrating lymphocytes (TILs) in a therapeutic population of TILs administered to a subject, wherein the frequency of unique TCR CDR3-encoding nucleic acid sequence clones identified in the first population of PBMCs as determined by RNA (including mRNA) sequencing is compared to the frequency of unique TCR CDR3-encoding nucleic acid sequence clones identified in the first population of PBMCs as determined by DNA sequencing, and wherein the frequency of such unique clones is greater as determined by RNA (including mRNA) sequencing.

Methods of the present invention for determining the persistence and activity of T cell receptor (TCR) complementarity determining region 3 (CDR3)-encoding nucleic acid sequence clones in tumor infiltrating lymphocytes (TILs) in a therapeutic population of TILs administered to a subject, may be applied to PBMCs isolated at various time after administration of a therapeutic population of TILs to a subject, for example about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 90 days, about 120 days, about 180 days, about 1 year, about 2 years, about 3 years, about 4 years, and/or about 5 years after administration of a therapeutic population of TILs.

### Systems for Identifying Clinically Effective Populations of TILs

In another aspect, the invention is directed to a system for identifying a clinically effective population of tumor infiltrating lymphocytes (TILs), the system comprising: memory; one or more processors; and one or more modules stored in memory and configured for execution by the one or more processors, the modules comprising instructions for:

(a) identifying the T cell receptor (TCR) complementarity determining region 3 (CDR3)-encoding nucleic acid sequence clones constituting the clonal diversity of a therapeutic population of TILs;

(b) identifying the T cell receptor (TCR) complementarity determining region 3 (CDR3)-encoding nucleic acid sequence clones constituting the clonal diversity of a first population of peripheral blood mononuclear cells (PBMCs), wherein the first population of PBMCs is isolated from a subject at least 14-days after the therapeutic population of step (a) is administered to said subject;

(c) for each unique TCR CDR3-encoding nucleic acid sequence clone identified in step (b), determining the frequency of such unique TCR CDR3 clone in each of the therapeutic population of TILs and the first population of PBMCs;

(d) sorting the unique TCR CDR3-encoding nucleic acid sequence clones identified in step (b) from highest frequency to lowest frequency for each of the therapeutic population of TILs and the first population of PBMCs; and,

(e) selecting the ten highest frequency unique TCR CDR3-encoding nucleic acid sequence clones from the first population of PBMCs sorted in step (d), wherein the TILs expressing such clones in the therapeutic population of TILs constitute a clinically effective sub-population of TILs, thereby identifying the clinically effective population of TILs.

In another aspect, the invention is directed to a system for identifying a clinically effective population of tumor infiltrating lymphocytes (TILs), the system further comprising a module comprising instructions for performing the steps of (i) identifying the TCR CDR3-encoding nucleic acid clones constituting the clonal diversity of a second population of PBMCs isolated from the subject prior to administration of the therapeutic population of TILs to the subject; and (ii) determining the frequency of each unique TCR CDR3-encoding nucleic acid sequence clone identified in step (i).

In another aspect, the invention is directed to a system for identifying a clinically effective population of tumor infiltrating lymphocytes (TILs), the system further comprising a module comprising instructions for comparing the TCR CDR3-encoding nucleic acid sequence clones constituting the CDR3 clonal diversity of the second population of PBMCs to the TCR CDR3-encoding nucleic acid sequence clones constituting the CDR3 clonal diversity of the first population of PBMCs.

In another aspect, the invention is directed to a system for identifying a clinically effective population of tumor infiltrating lymphocytes (TILs), the system further comprising a module comprising instructions for determining the frequency of unique TCR CDR3-encoding nucleic acid sequence clones in at least one population based on DNA sequence data.

In another aspect, the invention is directed to a system for identifying a clinically effective population of tumor infiltrating lymphocytes (TILs), the system further comprising a module comprising instructions for determining the frequency of unique TCR CDR3-encoding nucleic acid sequence clones in at least one population based on RNA sequence data.

In another aspect, the invention is directed to a system for identifying a clinically effective population of tumor infiltrating lymphocytes (TILs), the system further comprising a module comprising instructions for determining the frequency of unique TCR CDR3-encoding nucleic acid sequence clones in at least one population based on both RNA sequence data and DNA sequence data.

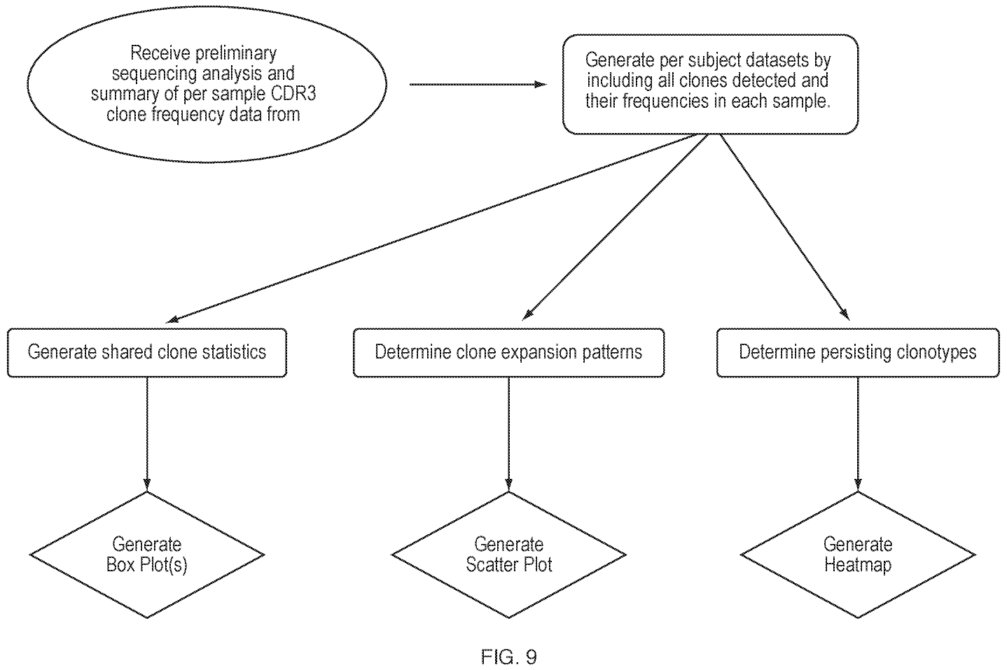

In another aspect, the invention is directed to a system for identifying a clinically effective population of tumor infiltrating lymphocytes (TILs), the system further comprising a module comprising instructions for comparing the frequency of unique TCR CDR3-encoding nucleic acid clones in the first population of PBMCs as determined by RNA sequencing to the frequency of unique TCR CDR3-encoding nucleic acid clones in the first population of PBMCs as determined by DNA sequencing, wherein the comparison is indicative of the clinically effective population of TILs. Exemplary modules, shown in FIG. 9, generate per subject datasets by including all clones detected and the clone frequencies in each sample. Results from this module may be processed by additional modules, also shown in FIG. 9, to generate shared clone statistics, determine clone expansion patterns, and determine persisting clonotypes. Further exemplary modules encode instructions to generate box, scatter, and heat maps based on the output from other modules of the system.

In another aspect, the invention is directed to a system for identifying a clinically effective population of tumor infiltrating lymphocytes (TILs), the system further comprising

at least one module, wherein the module encodes instructions for manipulating data obtained from TCR CDR3-encoding nucleic acid clones that are mRNA clones, wherein such data was determined by RNA sequencing.

### Systems for Persistence

In another aspect, the invention is directed to a system for determining the persistence and activity of T cell receptor (TCR) complementarity determining region 3 (CDR3)-encoding nucleic acid sequence clones in tumor infiltrating lymphocytes (TILs) in a therapeutic population of TILs administered to a subject, the system comprising: memory; one or more processors; and one or more modules stored in memory and configured for execution by the one or more processors, the modules comprising instructions for:

(a) identifying the T cell receptor (TCR) complementarity determining region 3 (CDR3)-encoding nucleic acid sequence clones constituting the clonal diversity of a therapeutic population of TILs;

(b) identifying the T cell receptor (TCR) complementarity determining region 3 (CDR3)-encoding nucleic acid sequence clones constituting the clonal diversity of a first population of peripheral blood mononuclear cells (PBMCs), wherein the first population of PBMCs is isolated from a subject at least 14-days after the therapeutic population of step (a) is administered to said subject;

(c) for each unique TCR CDR3-encoding nucleic acid sequence clone identified in step (b), determining the frequency of such unique TCR CDR3 clone in each of the therapeutic population of TILs and the first population of PBMCs; and

(d) for each unique TCR CDR3-encoding nucleic acid sequence clone identified in step (b), comparing the frequency of the TCR CDR3-encoding nucleic acid sequence clone in the first population of PBMCs to the frequency of the TCR CDR3-encoding nucleic acid sequence clone in the therapeutic population of TILs to determine the persistence and activity of TCR CDR3-encoding nucleic acid sequence clones in the therapeutic TIL population administered to the subject.

In another aspect, the invention is directed to the system for determining the persistence and activity of T cell receptor (TCR) complementarity determining region 3 (CDR3)-encoding nucleic acid sequence clones in tumor infiltrating lymphocytes (TILs) in a therapeutic population of TILs described above, modified to further comprise a module comprising instructions for determining the frequency of unique TCR CDR3-encoding nucleic acid sequence clones in at least one population based on DNA sequence data.

In another aspect, the invention is directed to any of the systems for determining the persistence and activity of T cell receptor (TCR) complementarity determining region 3 (CDR3)-encoding nucleic acid sequence clones in tumor infiltrating lymphocytes (TILs) in a therapeutic population of TILs described above, modified as applicable to further comprise a module comprising instructions for determining the frequency of unique TCR CDR3-encoding nucleic acid sequence clones in at least one population based on DNA sequence data.

In another aspect, the invention is directed to any of the systems for determining the persistence and activity of T cell receptor (TCR) complementarity determining region 3 (CDR3)-encoding nucleic acid sequence clones in tumor infiltrating lymphocytes (TILs) in a therapeutic population of TILs described above, modified as applicable to further comprise a module comprising instructions for determining the frequency of unique TCR CDR3-encoding nucleic acid sequence clones in at least one population based on RNA sequence data.

In another aspect, the invention is directed to any of the systems for determining the persistence and activity of T cell receptor (TCR) complementarity determining region 3 (CDR3)-encoding nucleic acid sequence clones in tumor infiltrating lymphocytes (TILs) in a therapeutic population of TILs described above, modified as applicable to further comprise a module comprising instructions for determining the frequency of unique TCR CDR3-encoding nucleic acid sequence clones in at least one population based on both RNA sequence data and DNA sequence data.

In another aspect, the invention is directed to any of the systems for determining the persistence and activity of T cell receptor (TCR) complementarity determining region 3 (CDR3)-encoding nucleic acid sequence clones in tumor infiltrating lymphocytes (TILs) in a therapeutic population of TILs described above, modified as applicable to further comprise a module comprising instructions for comparing the frequency of unique TCR CDR3-encoding nucleic acid clones in the first population of PBMCs as determined by RNA sequencing to the frequency of unique TCR CDR3-encoding nucleic acid clones in the first population of PBMCs as determined by DNA sequencing, wherein the comparison is indicative of the persistence and activity of TCR CDR3-encoding nucleic acid sequence clones in the therapeutic TIL population. Exemplary modules, shown in FIG. 9, generate per subject datasets by including all clones detected and the clone frequencies in each sample. Results from this module may be processed by additional modules, also shown in FIG. 9, to generate shared clone statistics, determine clone expansion patterns, and determine persisting clonotypes. Further exemplary modules encode instructions to generate box, scatter, and heat maps based on the output from other modules of the system.

### Tumor Types

In some embodiments, the methods of the present invention utilize initial TIL expanded from a tumor sample of a subject suffering from a cancer. The tumor sample may be obtained using methods known in the art, generally via surgical resection, needle biopsy or other means for obtaining a sample that contains a mixture of tumor and TIL cells. In general, the tumor sample may be from any solid tumor, including primary tumors, invasive tumors or metastatic tumors. The tumor sample may also be a liquid tumor, such as a tumor obtained from a hematological malignancy. The solid tumor may be of any cancer type, including, but not limited to, breast, pancreatic, prostate, colorectal, lung, brain, renal, stomach, and skin (including but not limited to squamous cell carcinoma, basal cell carcinoma, and melanoma).

In some embodiments, the subject suffers from a solid tumor cancer. In some embodiments, the solid tumor cancer is selected from the group consisting of melanoma (including uveal melanoma), ovarian cancer, cervical cancer, non-small-cell lung cancer (NSCLC), lung cancer, bladder cancer, breast cancer, pancreatic cancer, colorectal cancer, stomach cancer, squamous cell carcinoma, basal cell carcinoma, cancer caused by human papilloma virus, head and neck cancer (including head and neck squamous cell carcinoma (HNSCC)), brain cancer glioblastoma (including GBM), gastrointestinal cancer, renal cancer, and renal cell carcinoma. In some embodiments, the solid tumor is melanoma. In some embodiments, the solid tumor is cervical cancer.

In some embodiments, the subject suffers from a hematological malignancy or “liquid cancer.” Hematological malignancies include, but are not limited to, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, and non-Hodgkin's lymphomas. In some embodiments, the hematologial malignancy is CLL. In some embodiments, the hematological malignancy is AML.

It is understood that embodiments of the system of the invention may provide only some of the aspects disclosed herein.

While preferred embodiments of the invention are shown and described herein, such embodiments are provided by way of example only and are not intended to otherwise limit the scope of the invention. Various alternatives to the described embodiments of the invention may be employed in practicing the invention.

### Examples

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: uCDR3 Clonal Diversity in Melanoma Patients

Twenty-five metastatic melanoma patients receiving lifileucel (LN-144) in C-144-01 study of advanced metastatic melanoma were evaluated. The composition of the initial TIL products and the T cells circulating 42 days post-infusion were analyzed to assess uCDR3 clonal diversity, and TIL persistence.

TIL products are polyclonal preparations of autologous T cells, each T cell clone expresses a unique T cell receptor (TCR) that can be identified by its complementary determining region 3 (CDR3). CDR3 of TIL products and corresponding post-infusion peripheral blood samples were subjected to RNA-seq, using commercially-available iRepertoire technology (Huntsville, Ala.). For a description of iRepertoire technology see, for example. (i) Han, Jian, Method for Evaluating and Comparing Immunorepertoires, U.S. Pat. No. 9,012,148, and (ii) Han, Jian, Method for Evaluating and Comparing Immunorepertoires, U.S. Pat. No. 9,012,148, the contents of each of which is incorporated by reference herein in its entirety for all purposes.

PBMCs were collected from each patient before their personalized TIL therapeutic was administered. 42 days after the therapeutic TIL administration, patient PBMCs were again collected.

For each sample the T-cell receptor (TCR) CDR3 diversity of (1) the personalized TIL therapeutic; (2) the pre-treatment PMBCs; and (3) the PBMCs collected 42-days after TIL infusion was determined. For each individual cell population, nucleic acids were isolated and purified. The isolated mRNA was used to identify the uCDR3s in each sample.

The post-sequencing, secondary analysis tools were developed using Python version 3.6.3. All analyses and figures were generated in an Anaconda3 package and environment management system. The outline of the steps to produce the data in FIGS. 2 through 7 were as follows.

Raw sequencing data were first preliminarily analyzed to produce a per sample summary CDR3 clone frequency data based on the ray iRepertoire clonotype sequence data, then the following were determined: (1) Total read/CDR3 counts generated by sequencing; and (2) Count of unique CDR3 (uCDR3) clones, then computed the Shannon Diversity Index for the samples. See FIG. 2.

Next, the per-subject datasets were generated by the system: first each sample was normalized to a 10 million read baseline, then a comprehensive list of clones per subject and the associated uCDR3 frequencies were generated for each sample. Next, the system analyzed the shared clone statistics for each subject: the uCDR3 counts for each sample was confirmed; then the count of uCDR3 found in 2 samples (shared uCDR3) was determined. These are matched TIL product and PBMC samples for each subject. The sum of the frequencies of the shared uCDR3 in each sample was then calculated as well as the contribution of shared uCDR3 clones to total uCDR3 clones in each sample.

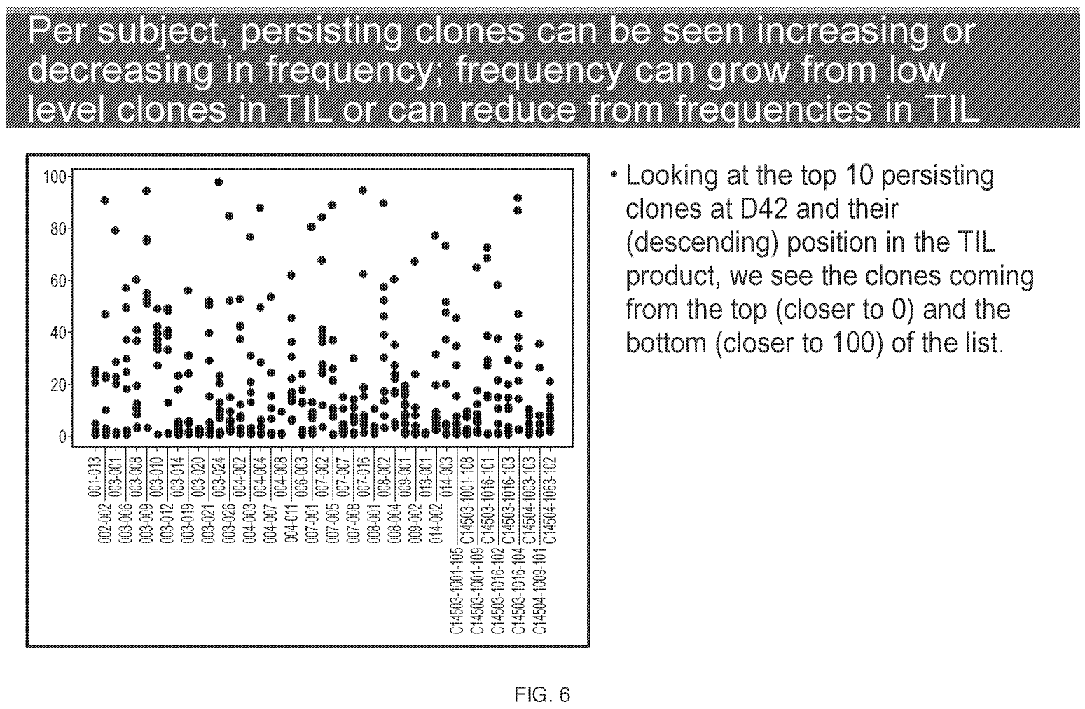

Next, uCDR3 clone expansion patterns were determined. First, the 10 most highly frequent shared clones Day 42 were identified. Then the percentile of each of the clones in the TIL Product, e.g. clone 1 of 100 will be in the 1%; clone 100 of 100 will be 100%. See FIG. 6. Finally, the system generated a scatter plot of all percentiles per subject.

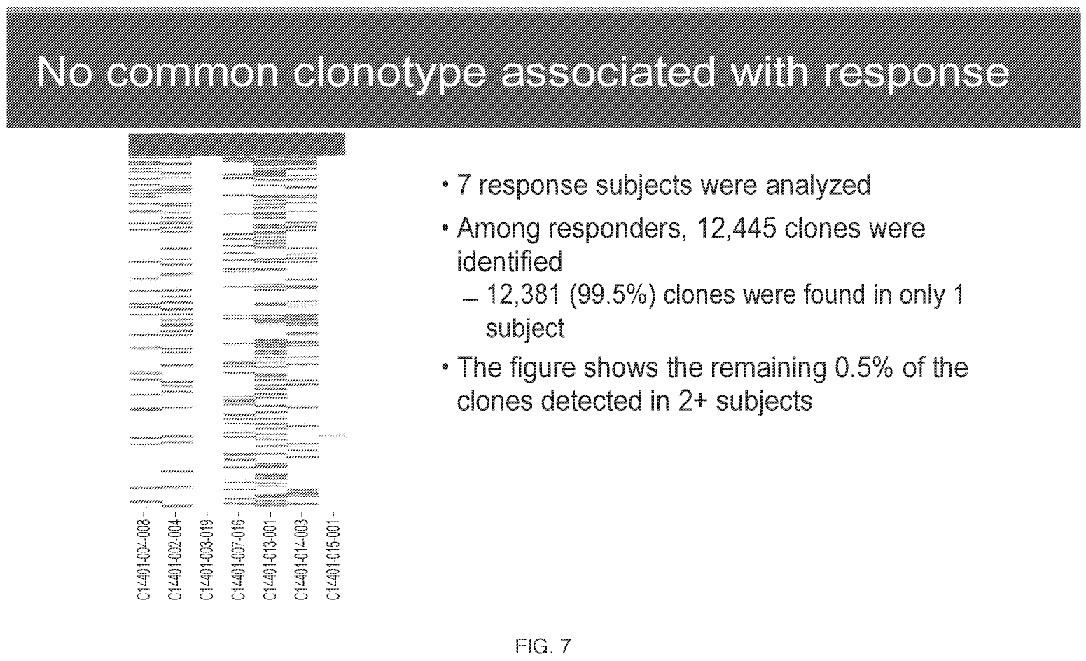

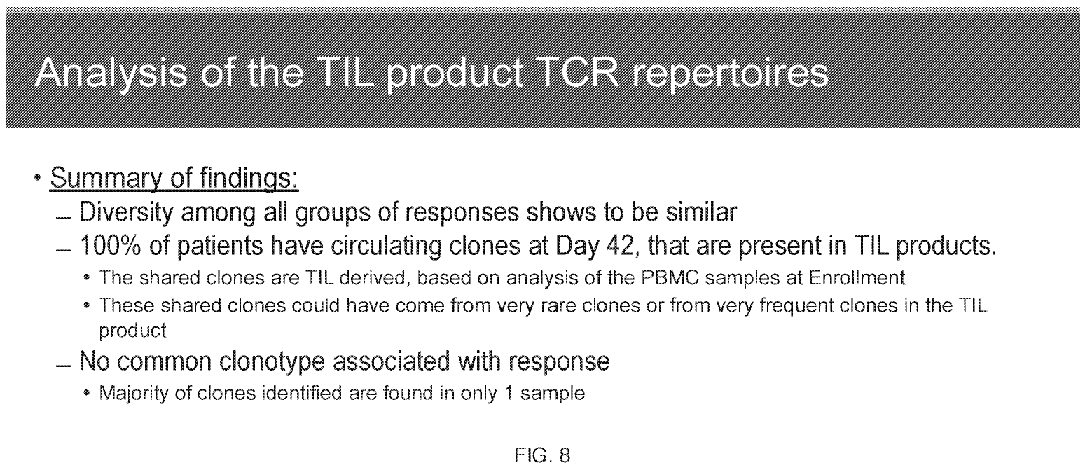

Next, the persistent uCDR3 clonotypes were determined. First, a comprehensive list of clones persistent in each subject was combined into an all-subject persistent clone list. Then an heatmap of clones vs. subjects highlighting persisting clones, see, e.g. FIG. 7.

Finally, using various modules of the system, comprehensive boxplots were produced to describe and characterize the total dataset, and included each of the following plots:
1. TIL Shannon Entropy (index)>Best Overall Response,
2. TIL unique CDR3 sequences (#)>Best Overall Response,
3. TIL unique CDR3 sequences (#)>Response,
4. TIL Shannon Entropy (index)>Response,
5. TIL shared portion>Response,
6. D42 unique CDR3 sequences (#)>Response,
7. D42 Shannon Entropy (Index)>Response,
8. D42 shared portion>Response,
9. Shared TIL & D42 uCDR3 (#)>Response,
10. Shared uCDR3 in TIL (%)>Response,
11. Shared uCDR3 in D42(%)>Response,
12. TIL unique CDR3 sequences (#)>Reduction on Day 42 Sum of Diameters,
13. TIL Shannon Entropy (index)>Reduction on Day 42 Sum of Diameters,
14. TIL shared portion>Reduction on Day 42 Sum of Diameters,
15. D42 unique CDR3 sequences (#)>Reduction on Day 42 Sum of Diameters,
16. D42 Shannon Entropy (Index)>Reduction on Day 42 Sum of Diameters,
17. D42 shared portion>Reduction on Day 42 Sum of Diameters,
18. Shared TIL & D42 uCDR3 (#)>Reduction on Day 42 Sum of Diameters,
19. Shared uCDR3 in TIL (%)>Reduction on Day 42 Sum of Diameters,
20. Shared uCDR3 in D42 (%)>Reduction on Day 42 Sum of Diameters,
21. TIL unique CDR3 sequences (#)>DCR,
22. TIL Shannon Entropy (index)>DCR,
23. TIL shared portion>DCR,
24. D42 unique CDR3 sequences (#)>DCR,
25. D42 Shannon Entropy (Index)>DCR,
26. D42 shared portion>DCR,
27. Shared TIL & D42 uCDR3 (#)>DCR,
28. Shared uCDR3 in TIL (%)>DCR,
29. Shared uCDR3 in D42(%)>DCR

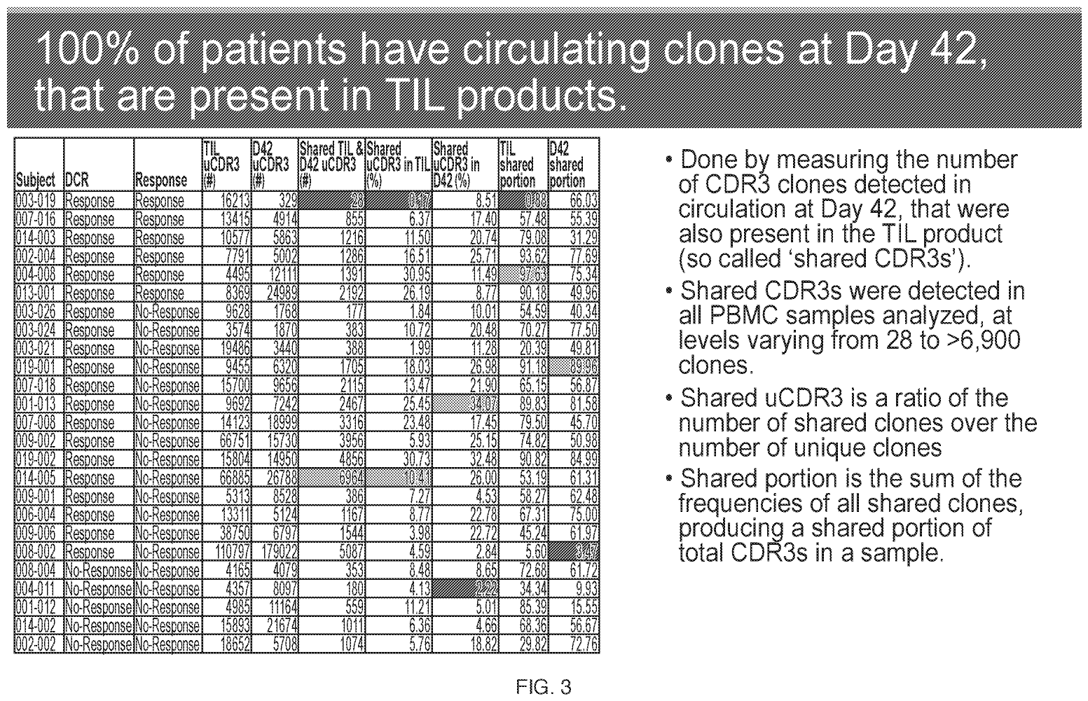

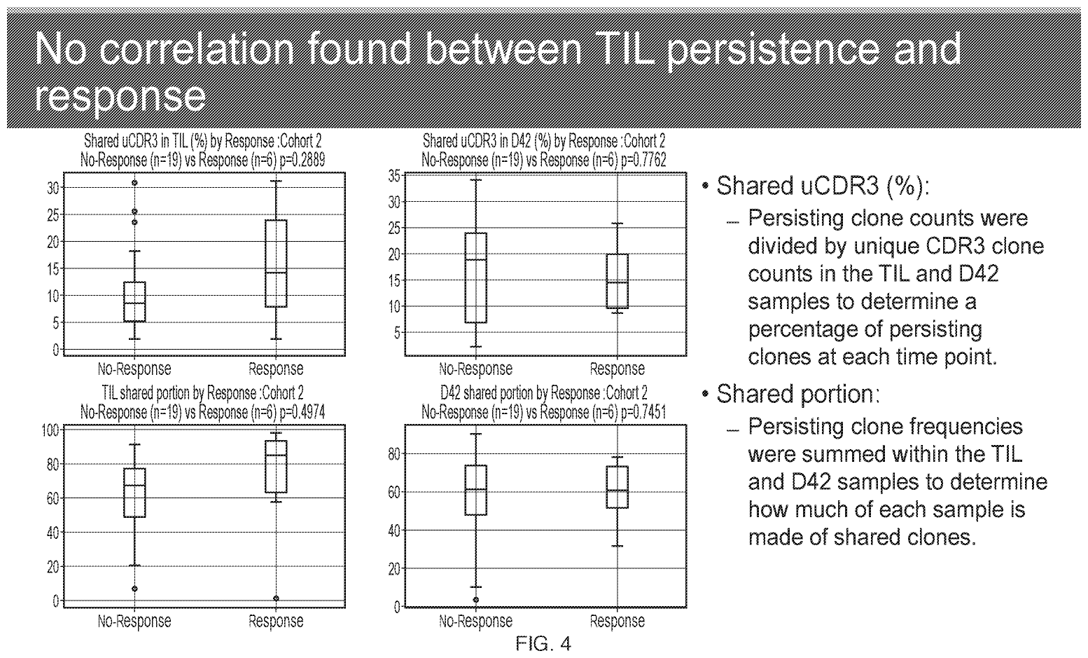

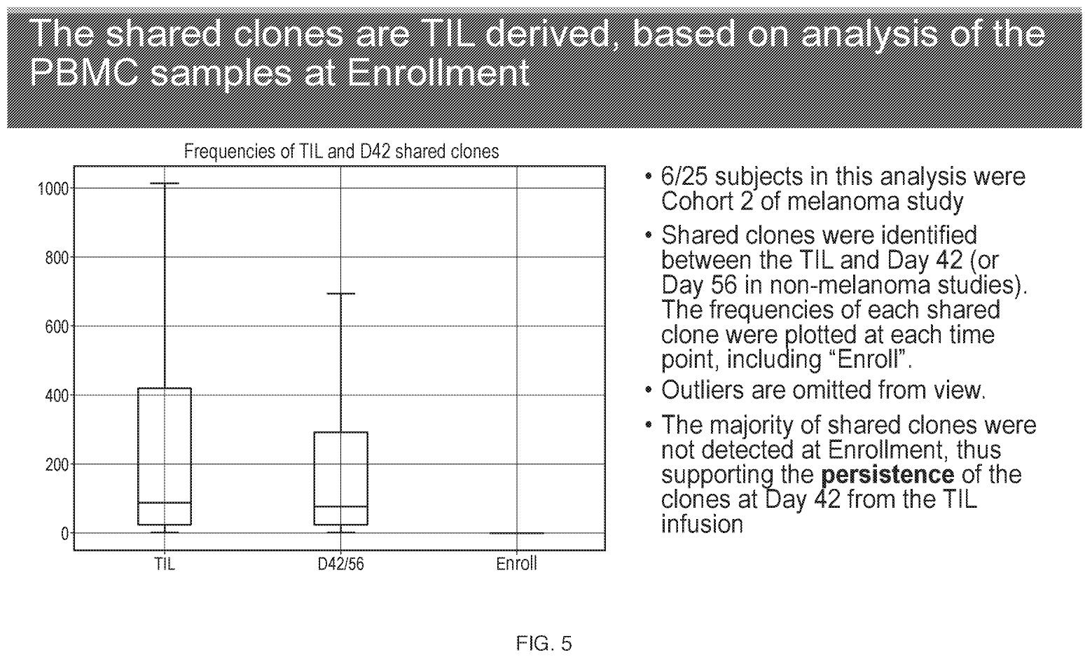

FIG. 2 shows the overall clonal diversity of patient not responding (n=28) (left distribution in each plot) and that of responding patients (n=10) (right distribution in each plot). A detailed summary of the frequency of uCDR3 clones shared between the TIL product and the Day 42 PBMCs is reported in FIG. 3. The average number of unique TCR CDR3 sequences (uCDR3) was 17511 [3574-110797] across TIL products, with Shannon diversity indexes varying from 2.7 to 10.8. Correlation analyses revealed no association between either parameter and clinical response, suggesting that tumor-reactive T cells are not affected by the level of diversity in bulk TIL (see, FIG. 4 and FIG. 5).

At 42 days post-infusion, TIL clones could be detected in the circulation of 100% of the patients, as depicted in FIG. 6. These Shared uCDR3 were found at levels varying from 28 to 6964 unique clonotypes and represented highly variable fractions of both the TIL product and the Day 42 circulating T cells.

The majority of Shared uCDR3 were not detected in the patients' peripheral blood at the time of enrollment, indicating that they represented intratumoral clonotypes that persisted after

TIL administration. In addition, shared uCDR3 clones represented at either high or low frequencies in the TIL product could persist for at least 6 weeks post-infusion. Finally, FIGS. 6 and 7 show that more than 97% of persisting clones were uniquely present in individual responding and non-responding patients, indicating a unique repertoire in each TIL preparation.

### Example 2: Persistence of Cryopreserved TIL Product in Advanced Melanoma Patients

Adoptive cell transfer utilizing tumor-infiltrating lymphocytes (TIL) is recognized as an effective treatment in metastatic melanoma and other solid tumors eliciting durable and complete responses in heavily pretreated patients, presumably by targeting somatic mutations specific to each tumor (Rosenberg et al. CCR 2011). Previously, patients with anti-PD-1-refractory advanced melanoma treated with lifileucel were reported to exhibit an overall response rate of 38% (SITC Nov2018). Here, the composition of the initial TIL products and the T cells circulating 42 days post-infusion (D42) were analyzed to uncover a potential link between clonal diversity, TIL in vivo persistence, and anti-tumor activity.

Since TIL products are preparations of polyclonal autologous T cells, each T cell clone expresses a unique T cell receptor (TCR) that can be identified by its complementary determining region 3 (CDR3). CDR3 of TIL products and corresponding post-infusion peripheral blood samples were subjected to RNA-seq, using iRepertoire technology (Huntsville, Ala.).

The average number of unique TCR CDR3 sequences (uCDR3) was 17511 [3574-110797] across TIL products, with Shannon diversity indexes varying from 2.7 to 10.8. Correlation analyses revealed no association between either parameter and clinical response, suggesting that tumor-reactive T cells may be present in low and high diversity bulk TIL products. At D42, TIL clones could be detected in the circulation of all treated patients. Numbers of Shared uCDR3 ranged from 28 to 6964 unique clonotypes and represented highly variable fractions of both the TIL product and the D42 circulating T cells. A link between Shared T cell clones and clinical response was previously hypothesized (Robbins et al. J Immunol 2004). In this study, comparable percentages of Shared uCDR3 were detected in responders and nonresponders. Importantly, the majority of Shared uCDR3 were not detected in the patients' peripheral blood at the time of enrollment, indicating that they represented intratumoral clonotypes that persisted after TIL administration. In addition, Shared uCDR3 clones represented at either high or low frequencies in the TIL product could persist for at least 6 weeks post-infusion. Finally, more than 97% of persisting clones were uniquely present in individual responding and non-responding patients, indicating a unique repertoire in each TIL preparation.

Overall, the data demonstrate that a fraction of TIL clones persisted in all patients. The uniqueness of the clonal profiles associated with response highlights the challenge of identifying a single TCR as mediator of activity and supports using a polyclonal product such as bulk TILs to treat solid tumors with their associated unique, patient-specific, mutational and neoantigen spectra.

### Example 3: uCDR3 Clonal Diversity in Cryopreserved TIL and Persistence of TIL in Advanced

### Metastatic Melanoma

Adoptive cell transfer utilizing tumor-infiltrating lymphocytes (TIL) is recognized as an effective treatment in metastatic melanoma and other solid tumors eliciting durable and complete responses in heavily pretreated patients, presumably by targeting somatic mutations specific to each tumor (Rosenberg et al. CCR 2011). Previously, patients with anti-PD-1-refractory advanced melanoma treated with lifileucel were reported to exhibit an overall response rate of 38% (SITC November 2018). Here, the composition of the initial TIL products and the T cells circulating 42 days post-infusion (D42) were analyzed to uncover a potential link between clonal diversity, TIL in vivo persistence, and anti-tumor activity.

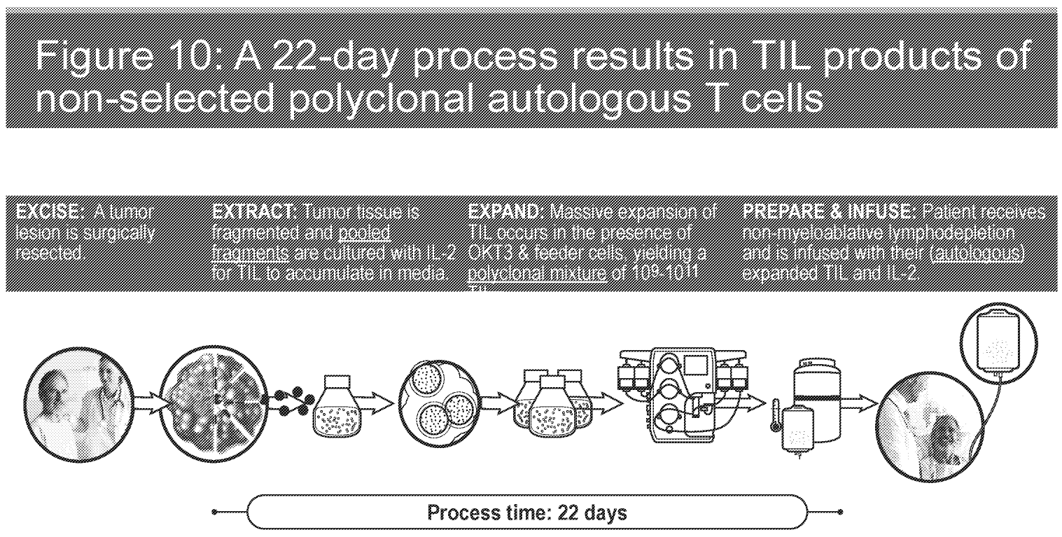

Clinical study C-144-01 is an ongoing Phase 2 multicenter study investigating autologous TIL (lifileucel, also called LN-144). The patient population includes patients with unresectable metastatic melanoma who have progressed on checkpoint inhibitors and BRAF/MEK inhibitors. TILs were harvested, expanded, and cryopreserved, and then prepared for infusion and infused into the patient in accordance with the 22-day process described in FIG. 10. A total of 27 matched pair samples (i.e., one TIL product sample and one D42 PBMC sample from the same patient) were analyzed.

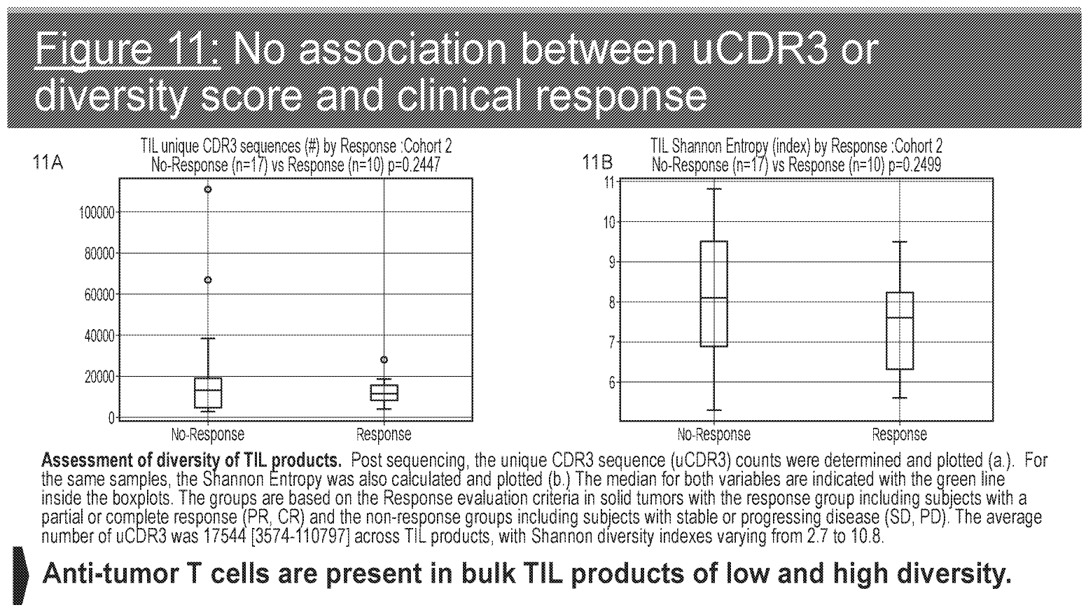

Since TIL products are preparations of polyclonal autologous T cells, each T cell clone expresses a unique T cell receptor (TCR) that can be identified by its complementary determining region 3 (CDR3). Total RNA was extracted using Qiagen RNeasy Mini Kit Protocol. CDR3 of TIL products and corresponding post-infusion peripheral blood samples were subjected to RNA amplification and sequencing using iRepertoire technology (Huntsville, Ala.). Custom python scripts were used to identify CDR3 clones of interest and perform statistical analyses. Post sequencing, the unique CDR3 sequence counts were calculated and plotted in FIG. 11A. Correlation analysis appeared to suggest no association between the number or diversity score of TCR clonotypes and clinical response. Nonresponders (n=17) and Responders (n=10) showed similar results (p=0.2447). For the same samples, the Shannon Entropy was also calculated and plotted in FIG. 11B. Again, nonresponders (n=17) and responders (n=10) showed similar results (p=0.2499). The median for both variables are indicated by the horizontal line in each of the boxplots. The groups are based on the response evaluation criteria in solid tumors with the response group including subjects with a partial or complete response and the non-response groups including subjects with stable or progressing disease. The average number of unique TCR CDR3 sequences (uCDR3) was 17511 [3574-110797] across TIL products, with Shannon diversity indexes varying from 2.7 to 10.8. Lack of correlation between the number of clonotypes and clinical response suggests that tumor-reactive T cells may be present in low and high diversity bulk TIL products. This confirms that bulk TIL products can recover relevant TIL without prior knowledge of tumor antigens.

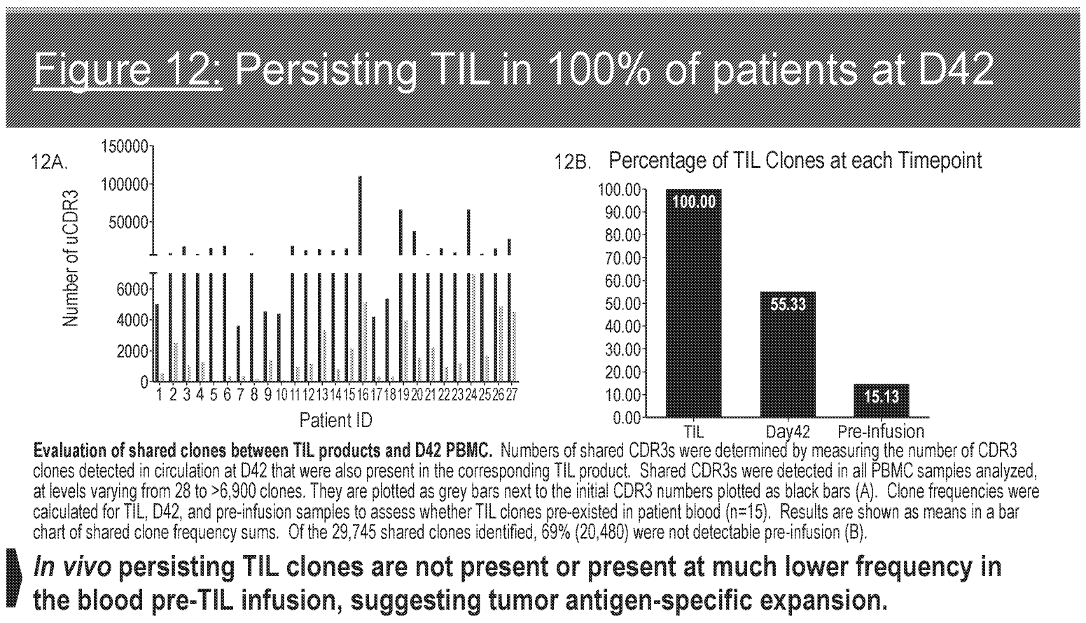

The number of shared CDR3s were determined by measuring the number of CDR3 clones detected in circulation (D42 samples) that were also present in the corresponding TIL product. Shared CDR3s were detecting in all D42 samples analyzed at levels varying from 28 to about 6900 clones. FIG. 12A illustrates the number of clones in the initial TIL product (black bars) and in the D42 samples (gray bars). Clone frequencies were calculated for TIL product, D42 samples, and pre-infusion PBMC to assess whether TIL clones pre-existed in patient blood. For this particular test, n=15. Results are shown in FIG. 12B as a bar chart of shared clone frequency sums expressed as a percentage. Of the 29,745 shared clones identified, 69% (or 20,480) were not detectable pre-infusion. This suggests that (i) in vivo persisting TIL clones are not present or present at much lower frequency in the blood pre-TIL infusion, and (ii) TIL product expansion is tumor antigen-specific.

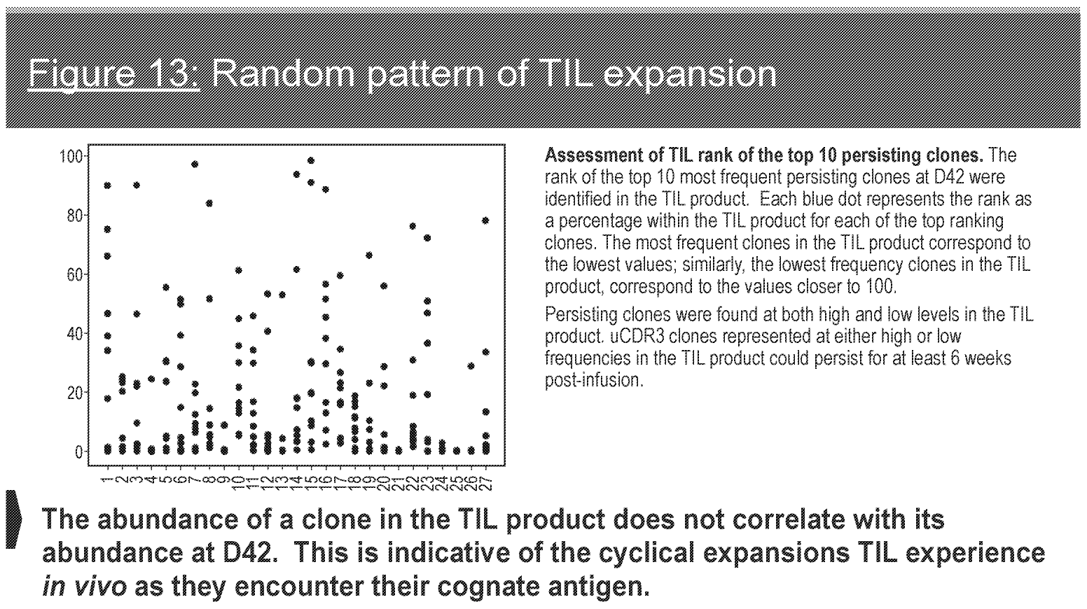

The top ten persisting clones were assessed and ranked in TIL product and D42 samples. Each dot in FIG. 13 illustrates the rank as a percentage within the TIL product for each of the top-ranking clones on a per-patient basis. The most frequent clones in the TIL product correspond to the lowest values; the lowest frequency clones correspond to the highest values (closer to 100). Persisting clones were found at both high and low frequency in the TIL product, and uCDR3 clones could persist for at least 6 weeks post-infsuion, whether high or low frequency in the TIL product. This data suggests that the abundance of a clone in the TIL product does not correlate with its abundance at D42 circulating levels in the blood. This is indicative of the cyclical expansions of TIL in vivo as they encounter their cognate antigen.

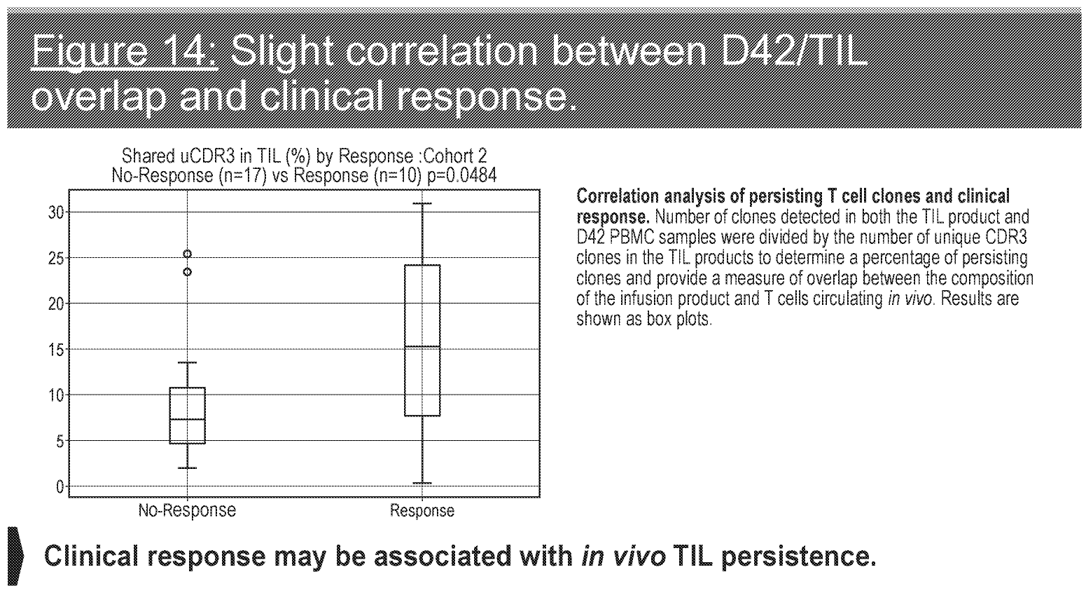

Correlation of persisting T-cell clones and clinical response was also analyzed. The number of clones detected in both TIL product and D42 samples were divided by the number of uCDR3 clones in the TIL products to determine a percentage of persisting clones. The measure of overlap, or shared uCDR3, (shown in %, p=0.0484) between the TIL product and the in vivo circulating T-cells at day 42 is shown as boxplots in FIG. 14 for nonresponders (n=17) and responders (n=10). The median for both variables are indicated by the horizontal line in each of the boxplots. This data suggest that clinical response may be associated with in vivo TIL persistence.

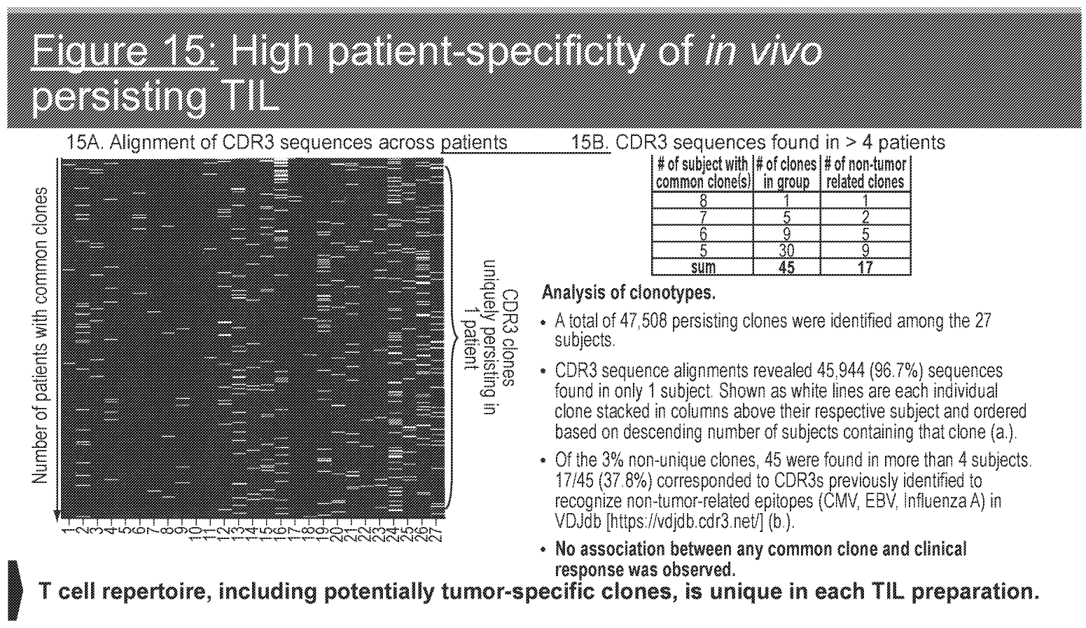

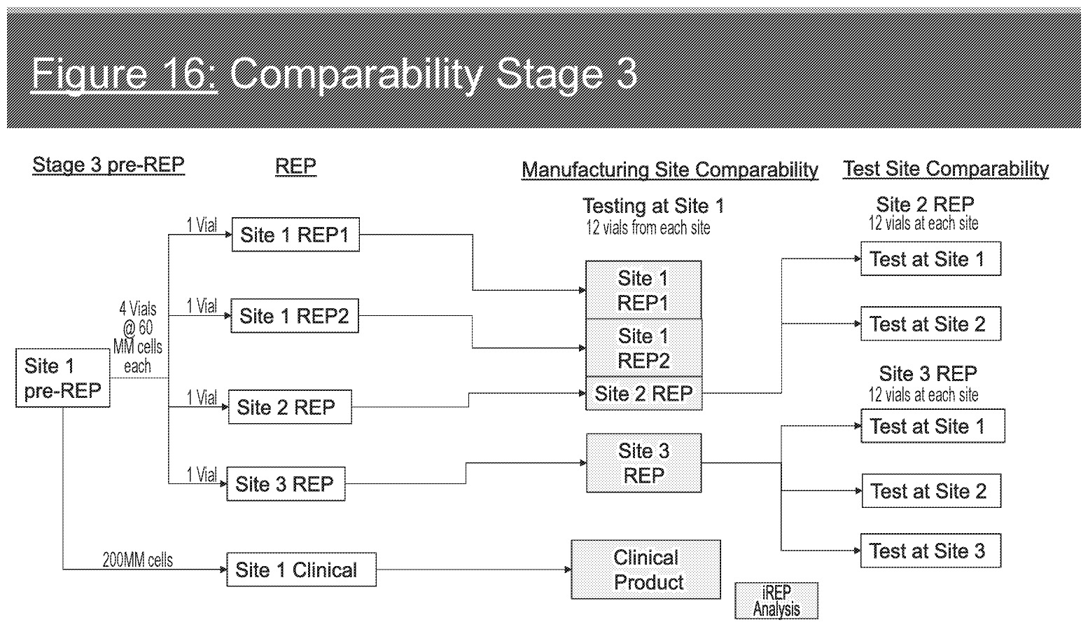

A total of 47,508 persisting clones were identified amonth the 27 subjects. CDR3 alignments revealed that 45,944 sequences (96.7%) were found in only 1 subject (see FIG. 15A). Forty-five sequences were found in more than 4 subjects; 17 of those (37.8%) corresponded to CDR3s previously identified to recognize non-tumor-related epiptopes (such as CMV, EBV, influenza, etc. (see https://vdjdb.cdr3.net). FIG. 15B illustrates this data in table form. The data suggest that there is no association between clonal commonality and clinical response, and that the T-cell repertoire, including potentially tumor-specific clones, is unique for each TIL preparation.

Overall, the data support using polyclonal product such as bulk TIL product to treat solid tumors with their associated unique, patient-specific, mutational and neoantigen spectra. The data demonstrate that 100% of the TIL products manufactured in accordance with the process described in FIG. 10 demonstrate substantial level of in vivo persistence at 6 weeks post-infusion. The TIL product is highly polyclonal, but the number of unique clones or diversity index are not related to clinical response. The in vivo fate of individual T-cell clones does not appear to depend on their frequency in the infusion product, reflective of their specific antigen reactivities. Further the TIL products are highly specific to each patient, and are comprised of unique TCR repertoires.

### Example 4: In Vivo Persistence of Tumor Infiltrating Lymphocyte (TIL) Product LN-145 in Cervical Cancer Patients

Clinical study C-145-04 (NCT03108495). is an ongoing Phase 2 multicenter study investigating autologous TIL (also called LN-145). Patients with metastatic, recurrent, or persistent cervical cancer were treated with an ex vivo expanded autologous TIL product (LN-145). The overall response rate was 44% and disease control rate was 85%.

The initial TIL in the pre-infusion LN-145 product and T cells circulating in the blood 42 days post-infusion (D42) were analyzed to uncover associations between clonal diversity, TIL in vivo persistence, and anti-tumor activity. Each T cell clone in a TIL product expresses a unique T cell receptor identifiable by its complementary determining region 3 (CDR3). Unique CDR3 sequences (uCDR3) and Shannon entropy for these T cell clones were identified using the methods discussed in the above examples. Initial LN-145 TIL from study C-145-04 and corresponding D42 post-infusion peripheral blood from the same patient were subjected to CDR3 RNA sequencing (iRepertoire, Huntsville, Ala.). The uCDR3 counts and Shannon diversity indices of pre-infusion LN-145 showed high variability—1,167 to 61,167 and 4.8 to 11.4, respectively—in both overall response rate and disease control rate cohorts, suggesting that both low and high diversity LN-145 may contain tumor-reactive T cells. In D42 peripheral blood samples, an average of 2079 LN-145-derived clones were present in all patients, representing 12% and 20% of LN-145 and D42 blood uCDR3s, respectively. These shared uCDR3s, however, represented a substantial portion of the total CDR3 repertoire of both LN-145 (62%) and D42 samples (52%). The overlap between pre-infusion LN-145 and D42 blood did not correlate with clinical response. Most clones common to LN-145 and D42 were not detected in peripheral blood at enrollment, indicating their likely LN-145 origin and post-infusion persistence. The frequency of each persisting clone in the initial TIL product did not predict prevalence in D42 blood. Finally, greater than 98% (32329/32757) of persisting clones were specific to individual responding and non-responding patients, indicating a unique repertoire in each TIL preparation.

Overall a fraction of TIL clones persisted in all patients of this highly responding population. The uniqueness of the clonal profile associated with each TIL product highlights the challenge of identifying a single TCR as a common mediator of activity and supports using polyclonal product such as bulk TIL to treat solid tumors with their associated private, patient-specific, mutational and neoantigen spectra.

### Example 5: Using TCR Repertoire Analysis to Determine TIL Production Process Comparability

Since TIL products are preparations of polyclonal autologous T cells, each T cell clone expresses a unique T cell receptor (TCR) that can be identified by its complementary determining region 3 (CDR3). The therapeutic efficacy of a TIL population can correlate with the CDR3 clonal diversity of the TILs in the population. However, variability, however minor, in the manufacturing processes from site to site can affect the CDR3 clonal diversity, and therefore the therapeutic efficacy, of a TIL population. Accordingly, a method of determining the comparability of TIL populations produced at different sites is desirable. In certain embodiments, the present disclosure provides a method of using TCR repertoire analysis to determine TIL production process comparability, the method comprising determining the CDR3 clonal diversity of TILs in a first sample, including a first set of unique CDR3 sequences expressed by the TILs in the first sample; determining the CDR3 clonal diversity of TILs in a second sample, including a second set of unique CDR3 sequences expressed by the TILs in the second sample; determining a number of unique CDR3 sequences occurring in both the first set and the second set; determining (i) a ratio of the number of unique CDR3 sequences occurring in both the first set and the second set to a number of the unique CDR3 sequences in the first set, and/or (ii) a ratio of the number of unique CDR3 sequences occurring in both the first set and the second set to a number of the unique CDR3 sequences in the second set; and based on said ratio, determining the comparability of the TILs in the first sample and the TILs in the second sample.

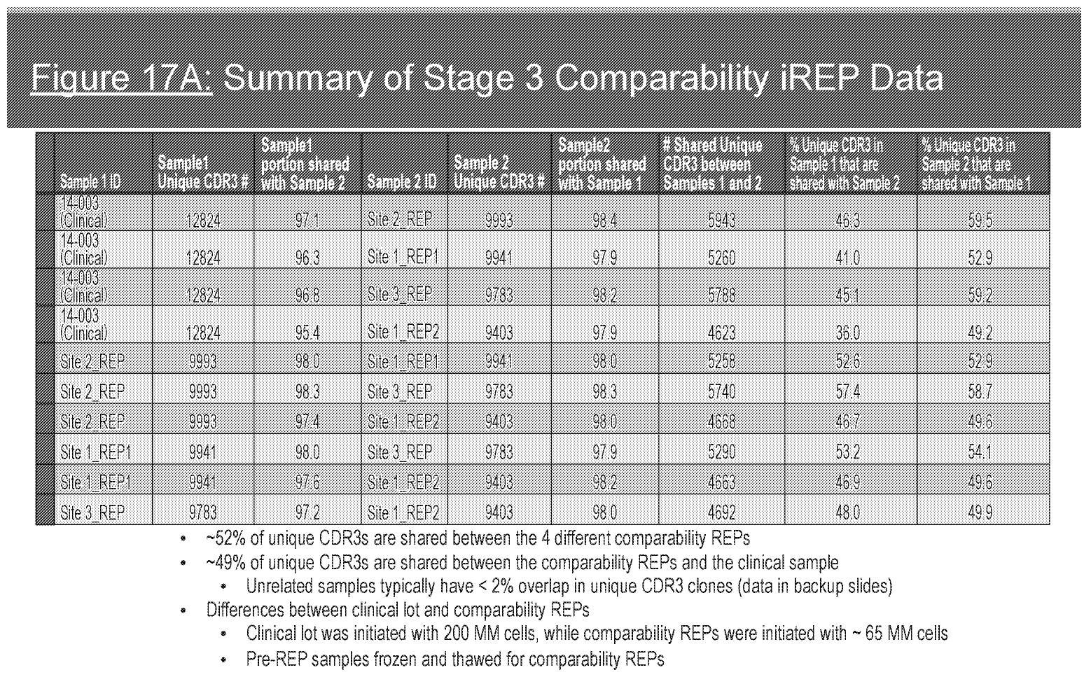

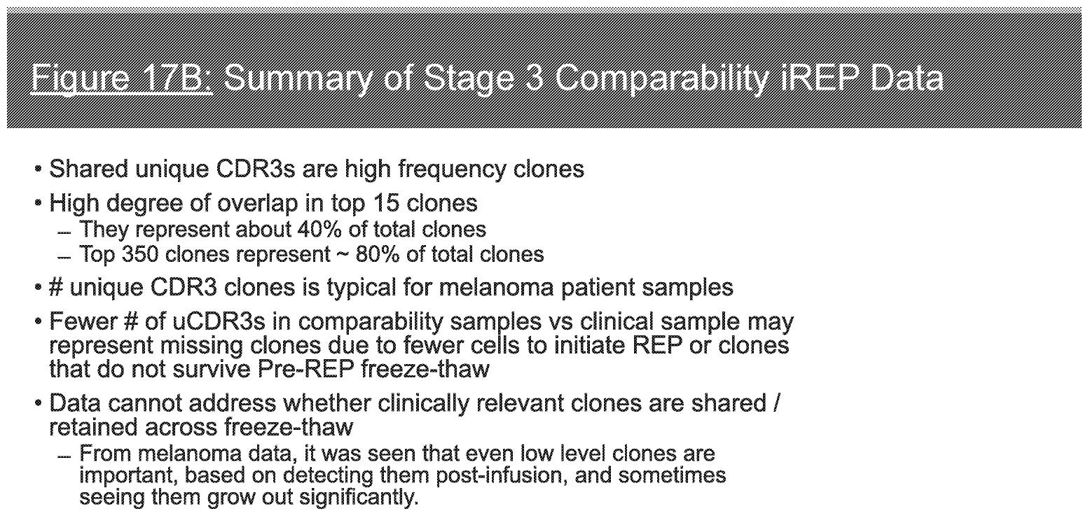

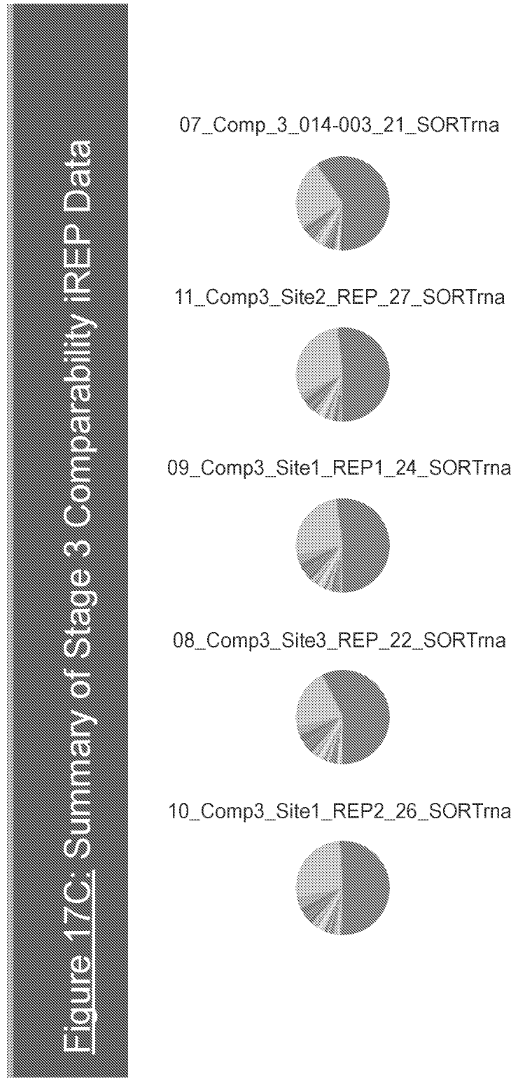

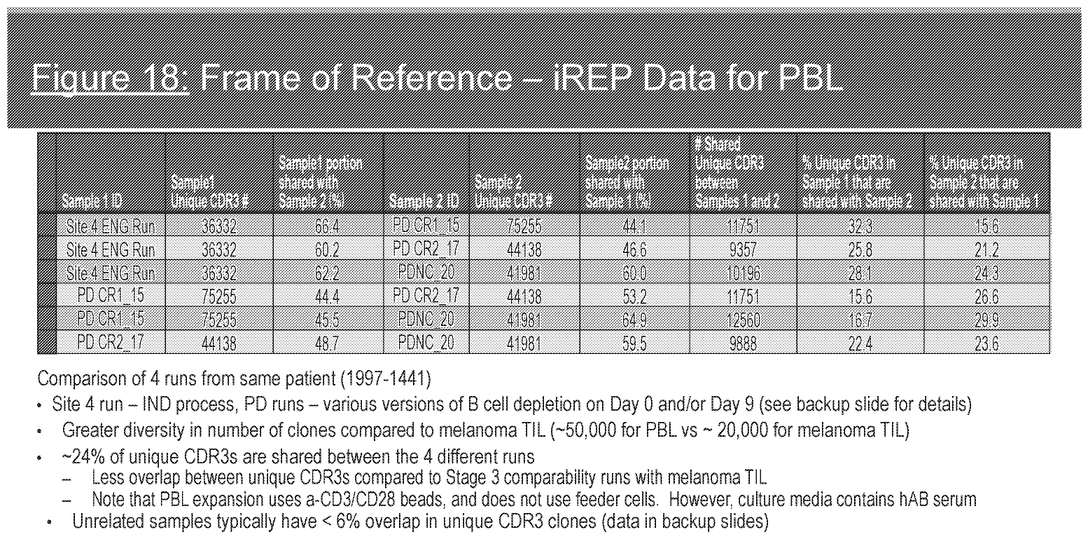

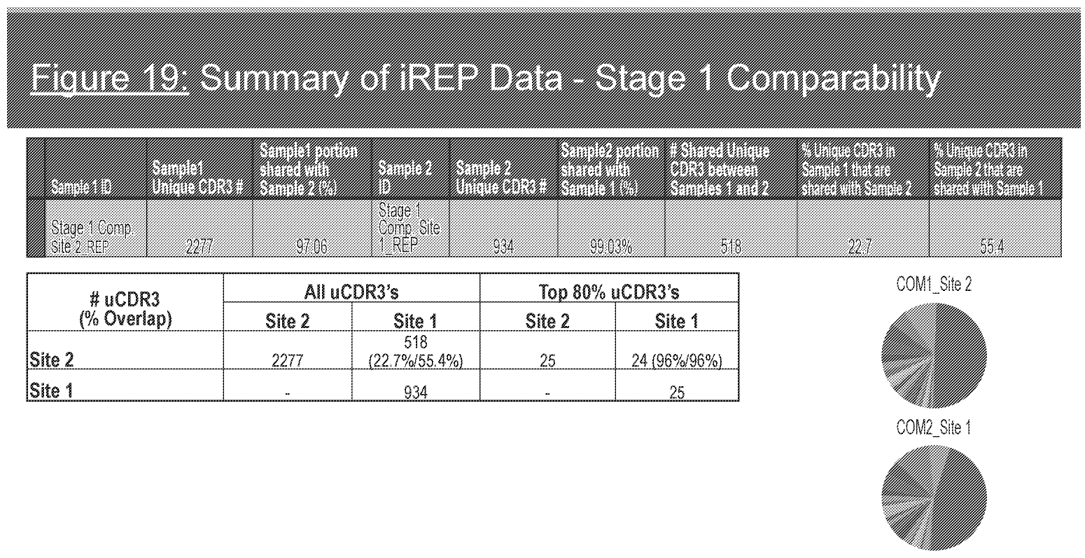

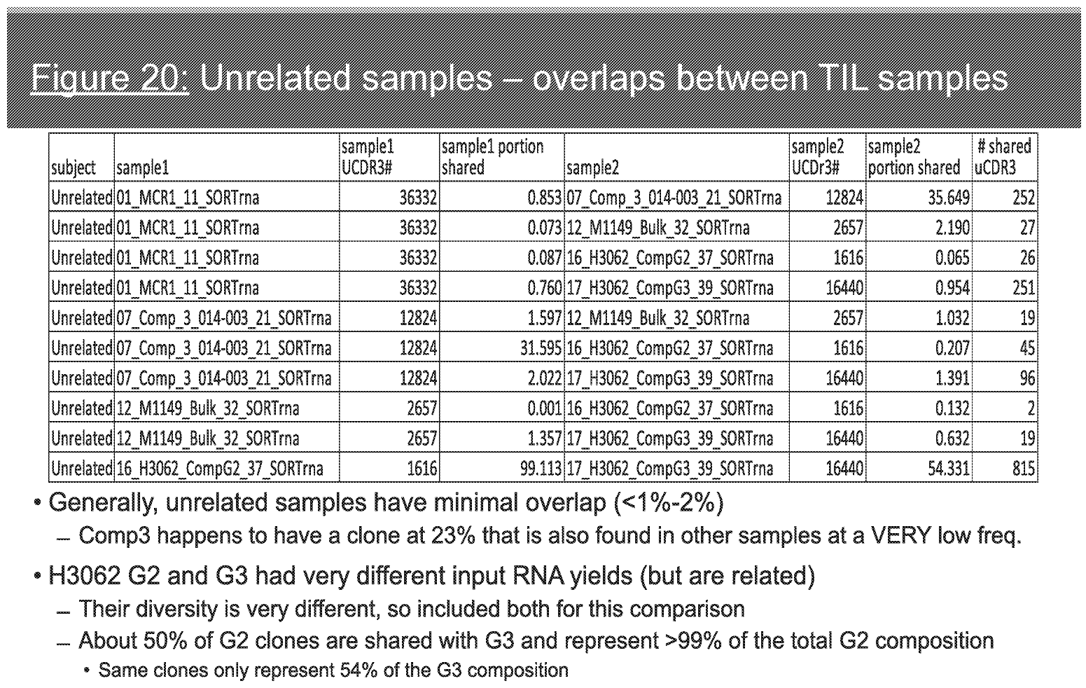

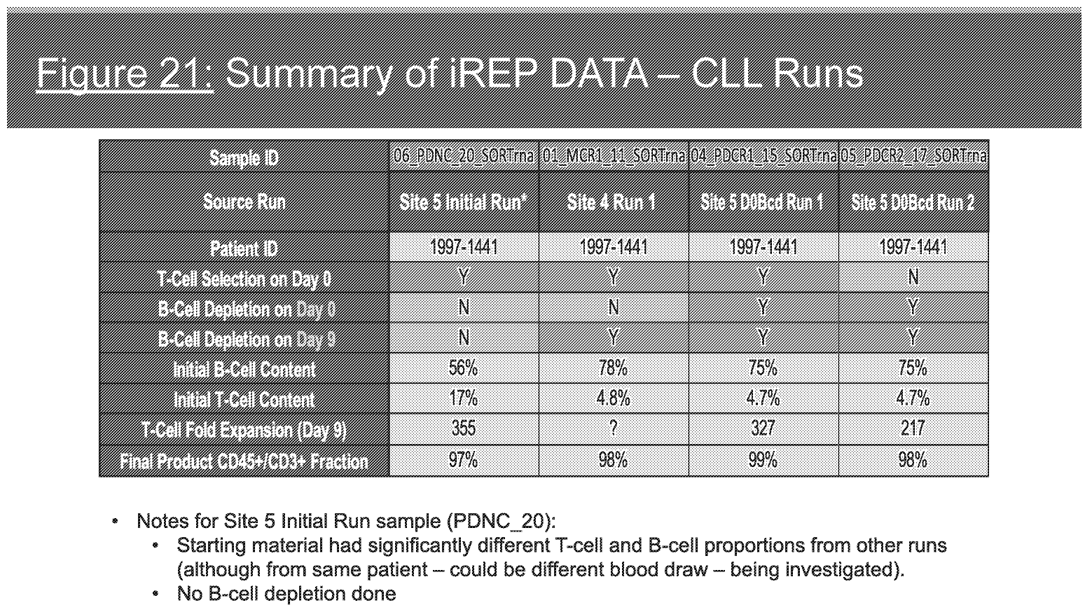

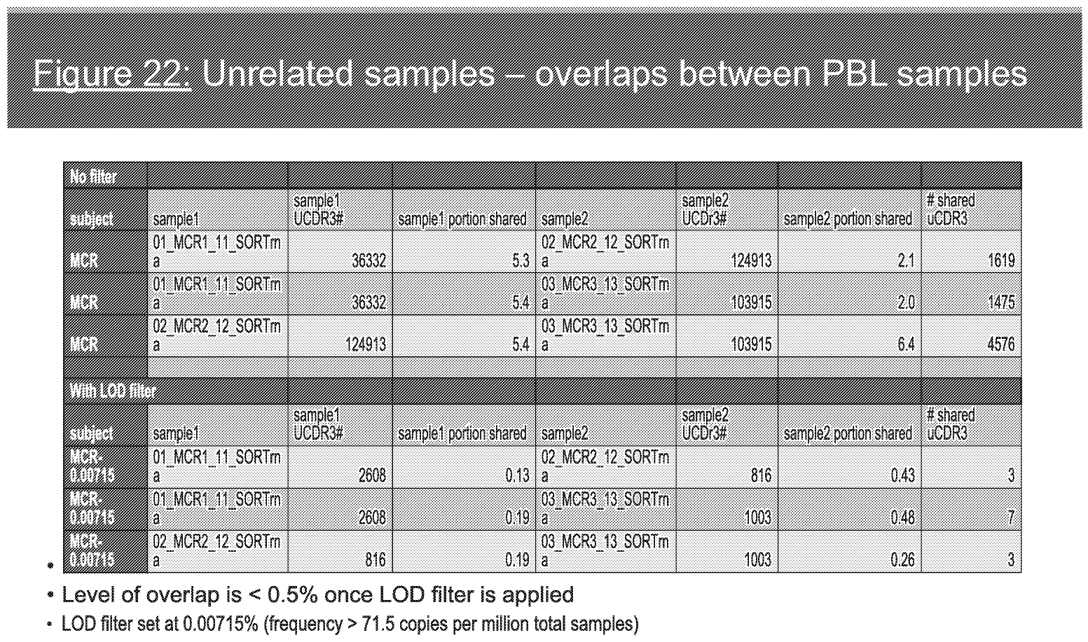

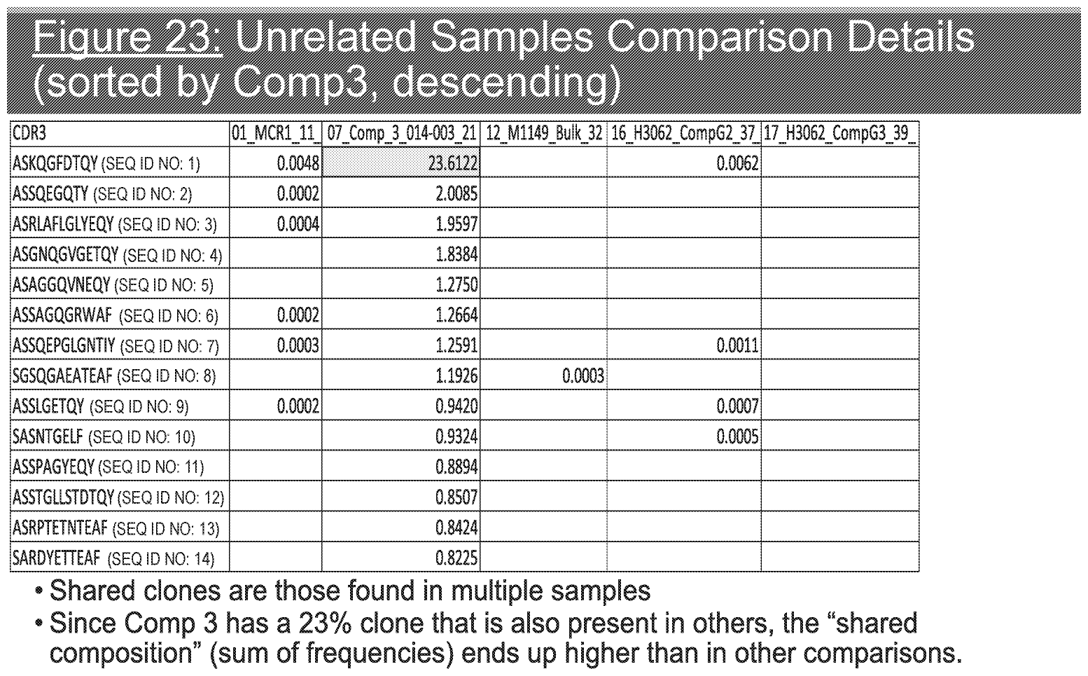

To test TIL production process comparability, T-cells undergo a pre-rapid expansion process followed by rapid expansion, as previously described, at 3 independent sites (FIG. 16). Total RNA was extracted from each sample using Qiagen RNeasy Mini Kit Protocol. CDR3 of TIL products were subjected to RNA amplification and sequencing using iRepertoire technology (Huntsville, Ala.). Custom python scripts were used to identify CDR3 clones of interest and perform statistical analyses. Post sequencing, the unique CDR3 sequence counts were calculated and shown in FIG. 17. To compare the CDR3 clonal diversity of the TILs in samples produced at different sites (e.g., a first sample and a second sample), a ratio of the number of unique CDR3 sequences shared by both samples to the number of unique CDR3 sequences in the first sample was determined. Similarly, a ratio of the number of unique CDR3 sequences shared by both samples to the number of unique CDR3 sequences in the second sample was also determined. The resulting value was a measure of the similarly of the CDR3 clonal diversity in the two samples, with a value of at least 10% or greater, in certain embodiments, indicating similarity in the CDR3 clonal diversity between the samples. As shown in FIG. 18, similar results were obtained when analyzing the iRepertoire data from peripheral blood lymphocyte (PBL) samples. Specifically, a ratio of the number of unique CDR3 sequences shared by samples produced at two different sites to the number of unique CDR3 sequences in either sample was determined to be greater than about 15%, indicating similarity in the CDR3 clonal diversity between the samples. In contrast, as shown in FIGS. 20, 22, and 23, a value of less than about 2% indicated that the samples were unrelated (e.g., samples having low comparability or similarity in CDR3 clonal diversity).

We claim:

1. A method for identifying a clinically effective population of tumor infiltrating lymphocytes (TILs) in a therapeutic population of TILs administered to a subject, the method comprising:
(i) identifying the T cell receptor (TCR) complementarity determining region 3 (CDR3)-encoding nucleic acid sequence clones constituting the TCR CDR3 clonal diversity of a therapeutic population of TILs;
(ii) identifying the TCR CDR3-encoding nucleic acid sequence clones constituting the TCR CD3 clonal diversity of a first population of peripheral blood mononuclear cells (PBMCs), wherein the first population of PBMCs is isolated from a subject at least 14-days after the therapeutic population of step (i) is administered to said subject;
(iii) for each unique TCR CDR3-encoding nucleic acid sequence clone identified in step (ii), determining the frequency of such unique TCR CDR3 clone in each of the therapeutic population of TILs and the first population of PBMCs;
(iv) sorting the unique TCR CDR3-encoding nucleic acid sequence clones identified in step (ii) from highest frequency to lowest frequency for each of the therapeutic population of TILs and the first population of PBMCs; and,
(v) selecting the ten highest frequency unique TCR CDR3-encoding nucleic acid sequence clones from the first population of PBMCs sorted in step (iv), wherein the TILs expressing such clones in the therapeutic population of TILs constitute a clinically effective population of TILs, thereby identifying the clinically effective population of TILs.

2. The method of claim 1, further comprising the steps of (a) identifying the TCR CDR3-encoding nucleic acid sequence clones constituting the TCR CDR3 clonal diversity of a second population of PBMCs isolated from the subject prior to administration of the therapeutic population of TILs to the subject; and (b) determining the frequency of each unique TCR CDR3-encoding nucleic acid sequence clone identified in step (a).

3. The method of claim 2, wherein the TCR CDR3-encoding nucleic acid sequence clones constituting the clonal diversity of the second population of PBMCs are different from the TCR CDR3-encoding nucleic acid sequence clones constituting the clonal diversity of the first population of PBMCs isolated from the subject post administration of the therapeutic population of TILs.

4. The method of any one of claims 1-3, wherein the frequency of unique TCR CDR3-encoding nucleic acid sequence clones identified in at least one population is determined by DNA sequencing.

5. The method of any one of claims 1-3, wherein the frequency of unique TCR CDR3-encoding nucleic acid sequence clones identified in at least one population is determined by RNA sequencing.

6. The method any one of claims 1-3, wherein the frequency of unique TCR CDR3-encoding nucleic acid sequence clones identified in the therapeutic population of TILs and the frequency of unique TCR CD3-encoding nucleic acid sequence clones identified in the first population of PBMCs are determined by both DNA and RNA sequencing.

7. The method of claim 6, wherein the frequency of unique TCR CDR3-encoding nucleic acid sequence clones identified in the first population of PBMCs as determined by RNA sequencing is compared to the frequency of unique TCR CDR3-encoding nucleic acid sequence clones identified in the first population of PBMCs as determined by DNA sequencing, wherein the frequency of such unique clones as determined by RNA sequencing is indicative of a clinically effective population of TILs as compared to the frequency of such unique clones as determined by DNA sequencing.

8. The method of claim 7, wherein the frequency of such unique clones is greater as determined by RNA sequencing.

9. The method of claim 7, wherein the frequency of such unique clones is greater as determined by DNA sequencing.

10. The method of claim 7, wherein the frequency of such unique clones as determined by RNA sequencing correlates to a population of TILs with enhanced therapeutic efficacy.

11. The method of claim 10, wherein the frequency of such unique clones as determined by DNA sequencing does not correlate to a population of TILs with enhanced therapeutic efficacy.

12. The method of claim 7, wherein the frequency of such unique clones as determined by RNA sequencing correlates to a population of TILs with enhanced therapeutic efficacy and the frequency of such unique clones as determined by DNA sequencing does not correlate to a population of TILs with enhanced therapeutic efficacy.

13. The method of any one of claims 1-12, wherein the TCR CDR3-encoding nucleic acid sequence clones are mRNA clones, which are identified by RNA sequencing.

14. The method of any one of claims 1-13, wherein the mRNA is capable of being detected at a period selected from the group consisting of about 20 days, about 25 days, about 30 days, about 35 days, about 40 days, about 42 days, about 45 days, about 50 days, about 55 days, about 60 days, about 90 days, about 120 days, about 180 days, about 1 year, and about 2 years post administration of a therapeutic population of TILs.

15. A method for enhancing a subject's T-cell repertoire, the method comprising:
(i) identifying a clinically effective population of tumor infiltrating lymphocytes according to the present disclosure; and
(ii) selecting and expanding the population identified in step (i) to produce a second a clinically effective therapeutic population of TILs.

16. The method of claim 15, the method further comprising the step of administering the expanded cells produced in step (ii), thereby enhancing the subject's T-cell repertoire.

17. A system for identifying a clinically effective population of tumor infiltrating lymphocytes (TILs), the system comprising:
memory;
one or more processors; and
one or more modules stored in memory and configured for execution by the one or more processors, the modules comprising instructions for:
(a) identifying the T cell receptor (TCR) complementarity determining region 3 (CDR3)-encoding nucleic acid sequence clones constituting the clonal diversity of a therapeutic population of TILs;
(b) identifying the T cell receptor (TCR) complementarity determining region 3 (CDR3)-encoding nucleic acid sequence clones constituting the clonal diversity of a first population of peripheral blood mononuclear cells (PBMCs), wherein the first population of PBMCs is isolated from a subject at least 14-days after the therapeutic population of step (a) is administered to said subject;
(c) for each unique TCR CDR3-encoding nucleic acid sequence clone identified in step (b), determining the frequency of such unique TCR CDR3 clone in each of the therapeutic population of TILs and the first population of PBMCs;
(d) sorting the unique TCR CDR3-encoding nucleic acid sequence clones identified in step (b) from highest frequency to lowest frequency for each of the therapeutic population of TILs and the first population of PBMCs; and,
(e) selecting the ten highest frequency unique TCR CDR3-encoding nucleic acid sequence clones from the first population of PBMCs sorted in step (d), wherein the TILs expressing such clones in the therapeutic population of TILs constitute a clinically effective sub-population of TILs, thereby identifying the clinically effective population of TILs.

18. The system of claim 17, further comprising a module comprising instructions for performing the steps of (x) identifying the TCR CDR3-encoding nucleic acid clones constituting the clonal diversity of a second population of PBMCs isolated from the subject prior to administration of the therapeutic population of TILs to the subject; and (y) determining the frequency of each unique TCR CDR3-encoding nucleic acid sequence clone identified in step (x).

19. The system of claim 17, further comprising a module comprising instructions for comparing the TCR CDR3-encoding nucleic acid sequence clones constituting the CDR3 clonal diversity of the second population of PBMCs before to the TCR CDR3-encoding nucleic acid sequence clones constituting the CDR3 clonal diversity of the first population of PBMCs.

20. The system of any one of claims 17-19, further comprising a module comprising instructions for determining the frequency of unique TCR CDR3-encoding nucleic acid sequence clones in at least one population based on DNA sequence data.

21. The system of any one of claims 17-19, further comprising a module comprising instructions for determining the frequency of unique TCR CDR3-encoding nucleic acid sequence clones in at least one population based on RNA sequence data.

22. The system of any one of claims 17-19, further comprising a module comprising instructions for determining the frequency of unique TCR CDR3-encoding nucleic acid sequence clones in at least one population based on both RNA sequence data and DNA sequence data.

23. The system of claim 22, further comprising a module comprising instructions for comparing the frequency of unique TCR CDR3-encoding nucleic acid clones in the first population of PBMCs as determined by RNA sequencing to the frequency of unique TCR CDR3-encoding nucleic acid clones in the first population of PBMCs as determined by DNA sequencing, wherein the comparison is indicative of the clinically effective population of TILs.

24. The system of any one of claims 17-23, wherein the TCR CDR3-encoding nucleic acid clones are mRNA clones, which are identified by RNA sequencing.

25. A method for determining the persistence and activity of T cell receptor (TCR) complementarity determining region 3 (CDR3)-encoding nucleic acid sequence clones in tumor infiltrating lymphocytes (TILs) in a therapeutic population of TILs administered to a subject, the method comprising:
(a) identifying the TCR CDR3-encoding nucleic acid clones constituting the TCR CDR3 clonal diversity of a therapeutic population of TILs;
(b) identifying the TCR CDR3-encoding nucleic acid sequence clones constituting the TCR CDR3 clonal diversity of a first population of peripheral blood mononuclear cells (PBMCs), wherein the first population of PBMCs is isolated from a subject at least 14-days after the therapeutic population of step (a) is administered to said subject;
(c) for each unique TCR CDR3-encoding nucleic acid sequence clone identified in step (b), determining the frequency of such unique TCR CDR3-encoding nucleic acid sequence clone in each of the therapeutic population of TILs and the first population of PBMCs; and
(d) for each unique TCR CDR3-encoding nucleic acid sequence clone identified in step (b), comparing the frequency of the TCR CDR3-encoding nucleic acid sequence clone in the first population of PBMCs to the frequency of the TCR CDR3-encoding nucleic acid sequence clone in the therapeutic population of TILs to determine the persistence and activity of TCR CDR3-encoding nucleic acid sequence clones in the therapeutic TIL population administered to the subject.

26. The method of claim 25, wherein the frequency of unique TCR CDR3-encoding nucleic acid sequence clones identified in the therapeutic population of TILs and the frequency of unique TCR CDR3-encoding nucleic acid sequence clones identified in the first population of PBMCs are determined by both DNA and RNA sequencing.

27. The method of claim 26, wherein the frequency of unique TCR CDR3-encoding nucleic acid sequence clones identified in the first population of PBMCs as determined by RNA sequencing is compared to the frequency of unique TCR CDR3-encoding nucleic acid sequence clones identified in the first population of PBMCs as determined by DNA sequencing, wherein the frequency of such unique clones as determined by RNA sequencing compared to the frequency of such unique clones as determined by DNA sequencing is indicative of the persistence and activity of such clones.

28. The method of claim 27, wherein the frequency of such unique clones is greater as determined by RNA sequencing.

29. A method for identifying a clinically effective population of tumor infiltrating lymphocytes (TILs) in a subject administered a therapeutic population of TILs, the method comprising:
(i) determining the CDR3 clonal diversity of a therapeutic population of TILs;
(ii) determining the CDR3 clonal diversity of peripheral blood mononuclear cells (PBMCs), wherein the PBMCs are isolated from a subject at least 14-days after the therapeutic population of step (i) is administered to said subject;
(iii) identifying the CDR3 clones identified in both steps (i) and (ii);
(iv) sorting the CDR3 clones identified in step (iii) from highest frequency to lowest frequency for each of the therapeutic population of TILs and the PBMCs; and,
(iv) selecting the ten highest frequency CDR3 clones from step (iv) identified in the PBMCs, thereby identifying the clinically effective population of TILs.

30. A system for identifying a clinically effective population of tumor infiltrating lymphocytes (TILs), the system comprising:
memory;
one or more processors; and
one or more modules stored in memory and configured for execution by the one or more processors, the modules comprising instructions for:
(a) determining the CDR3 clonal diversity of a therapeutic population of TILs;
(b) determining the CDR3 clonal diversity of peripheral blood mononuclear cells (PBMCs), wherein the PBMCs are isolated from a subject at least 14-days after the therapeutic population of step (a) is administered to said subject;
(c) identifying the CDR3 clones identified in both steps (a) and (b);
(d) sorting the CDR3 clones identified in step (c) from highest frequency to lowest frequency for each of the therapeutic population of TILs and the PBMCs; and,
(e) selecting the ten highest frequency CDR3 clones from step (d) identified in the PBMCs, in order to identify a clinically effective population of TILs.

31. The method of any of claims 1-30, wherein the subject has a solid tumor cancer.

32. The method of claim 31, wherein the solid tumor cancer is selected from the group consisting of melanoma (including uveal melanoma), ovarian cancer, cervical cancer, non-small-cell lung cancer (NSCLC), lung cancer, bladder cancer, breast cancer, pancreatic cancer, colorectal cancer, stomach cancer, squamous cell carcinoma, basal cell carcinoma, cancer caused by human papilloma virus, head and neck cancer (including head and neck squamous cell carcinoma (HNSCC)), brain cancer glioblastoma (including GBM), gastrointestinal cancer, renal cancer, and renal cell carcinoma.

33. The method of claim 31, wherein the cancer is melanoma.

34. The method of claim 31, wherein the cancer is cervical cancer.

35. A method of using TCR repertoire analysis to determine TIL production process comparability, the method comprising:
determining a first set of unique CDR3 sequences expressed by the TILs in the first sample;
determining a second set of unique CDR3 sequences expressed by the TILs in the second sample;
determining (i) a ratio of the number of unique CDR3 sequences occurring in both the first set and the second set to a number of the unique CDR3 sequences in the first set, and/or (ii) a ratio of the number of unique CDR3 sequences occurring in both the first set and the second set to a number of the unique CDR3 sequences in the second set; and
based on said ratio, determining the comparability of the TILs in the first sample and the TILs in the second sample.

36. The method of claim 35, wherein the TILs of the first sample are produced at a first site, and wherein the TILs of the second sample are produced at a second site.

37. The method of any one of claims 35-36, wherein the number of unique CDR3 sequences in the first set correlates to the therapeutic efficacy of the TILs in the first sample.

38. The method of any one of claims 35-36, wherein the number of unique CDR3 sequences in the second set correlates to the therapeutic efficacy of the TILs in the second sample.

39. The method of claim 35, wherein the first sample and the second sample are derived from the same sample.

40. The method of any one of claims 35-39, wherein at least one of the first sample and the second sample are obtained from a subject having a solid tumor cancer.

41. The method of claim 40, wherein the solid tumor cancer is selected from the group consisting of melanoma (including uveal melanoma), ovarian cancer, cervical cancer, non-small-cell lung cancer (NSCLC), lung cancer, bladder cancer, breast cancer, pancreatic cancer, colorectal cancer, stomach cancer, squamous cell carcinoma, basal cell carcinoma, cancer caused by human papilloma virus, head and neck cancer (including head and neck squamous cell carcinoma (HNSCC)), brain cancer glioblastoma (including GBM), gastrointestinal cancer, renal cancer, and renal cell carcinoma.

42. The method of claim 41, wherein the solid tumor cancer is melanoma.

43. The method of claim 41, wherein the solid tumor cancer is cervical cancer.

44. The method of any one of claims 35-43, wherein the TILS in the first sample and the TILs in the second sample are post-rapid expansion process (REP) TILs.

45. The method of any one of claims 35-44, wherein the ratio having a value of about 0.4 or greater, about 0.42 or greater, about 0.44 or greater, about 0.46 or greater, about 0.48 or greater, about 0.50 or greater, about 0.52 or greater, about 0.54 or greater, about 0.56 or greater, about 0.58 or greater, or about 0.60 or greater indicates comparability between the first sample and the second sample.

46. The method of any one of claims 35-45, wherein the CDR3 clonal diversity of TILs in the first sample and/or the second sample is determined by DNA sequencing.

47. The method of any one of claims 35-45, wherein the CDR3 clonal diversity of TILs in the first sample and/or the second sample is determined by RNA sequencing.

48. The method of any one of claims 35-47, wherein the first set of unique CDR3 sequences consists of a given number of CDR3 sequences expressed at the highest frequency by the TILs in the first sample.

49. The method of any one of claims 35-48, wherein the second set of unique CDR3 sequences consists of a given number of CDR3 sequences expressed at the highest frequency by the TILs in the second sample.

50. The method of any one of claims 48 and 49, wherein the given number is selected from the group consisting of about 5, about 10, about 15, about 20, about 25, about 50, about 75, about 100, about 150, about 200, about 250, about 300, about 350, about 400, about 450, about 500, and greater than about 500.

51. The method of any one of claims 48 and 49, wherein the given number correlates to greater than about 10%, greater than about 20%, greater than about 30%, greater than about 40%, greater than about 50%, greater than about 60%, greater than about 70%, greater than about 80%, greater than about 90%, or about 100% of a total number of sequences in one or both of the first set of unique CDR3 sequences and the second set of unique CDR3 sequences.

52. The method of any one of claims 48 and 49, wherein the given number is between about 10 and about 20.

53. The method of claim 52, wherein the given number correlates to about 40% of a total number of sequences in one or both of the first set of unique CDR3 sequences and the second set of unique CDR3 sequences.

54. The method of any one of claims 48 and 49, wherein the given number is between about 300 and about 400.

55. The method of claim 52, wherein the given number correlates to about 80% of a total number of sequences in one or both of the first set of unique CDR3 sequences and the second set of unique CDR3 sequences.


SEQUENCE LISTING (14 sequences)

SEQ ID NO: 1 - protein, 10 aa, Homo sapiens
ASKQGFDTQY

SEQ ID NO: 2 - protein, 9 aa, Homo sapiens
ASSQEGQTY

SEQ ID NO: 3 - protein, 13 aa, Homo sapiens
ASRLAFLGLYEQY

SEQ ID NO: 4 - protein, 12 aa, Homo sapiens
ASGNQGVGETQY

SEQ ID NO: 5 - protein, 11 aa, Homo sapiens
ASAGGQVNEQY

SEQ ID NO: 6 - protein, 11 aa, Homo sapiens
ASSAGQGRWAF

SEQ ID NO: 7 - protein, 13 aa, Homo sapiens
ASSQEPGLGNTIY

SEQ ID NO: 8 - protein, 12 aa, Homo sapiens
SGSQGAEATEAF

SEQ ID NO: 9 - protein, 9 aa, Homo sapiens
ASSLGETQY

SEQ ID NO: 10 - protein, 9 aa, Homo sapiens
SASNTGELF

SEQ ID NO: 11 - protein, 10 aa, Homo sapiens
ASSPAGYEQY

SEQ ID NO: 12 - protein, 13 aa, Homo sapiens
ASSTGLLSTDTQY

SEQ ID NO: 13 - protein, 12 aa, Homo sapiens
ASRPTETNTEAF

SEQ ID NO: 14 - protein, 11 aa, Homo sapiens
SARDYETTEAF